(19) United States
(12) Patent Application Publication
(10) Pub. No.: US 2006/0036094 A1
(43) Pub. Date: Feb. 16, 2006

(54) Method for producing zoanthamine alkaloid and intermediate used in same

(76) Inventor: Masaaki Miyashita, Sapporo-shi (JP)

Correspondence Address: HARNESS, DICKEY & PIERCE, P.L.C., P.O. BOX 8910, RESTON, VA, 20195, US

(21) Appl. No.: 11/059,359

(22) Filed: Feb. 17, 2005

(30) Foreign Application Priority Data
Aug. 16, 2004 (JP) 2004-236445

(51) Int. Cl. A61K 31/4745; C07D 491/04

(52) U.S. Cl. 546/41; 549/356

(57) ABSTRACT

The present invention provides a method for producing a zoanthamine alkaloid and an intermediate suitably used in the method. The method realizes high-yield synthesis of a zoanthamine alkaloid such as norzoanthamine or the like.

This Nonprovisional application claims priority under 35 U.S.C. § 119(a) on Patent Application No. 2004/236445filed in Japan on Aug. 16, 2004, the entire contents of which are hereby incorporated by reference.

### FIELD OF THE INVENTION

This invention relates to a method for producing a zoanthamine alkaloid and an intermediate used in the method. The present invention is particularly suitable for producing norzoanthamine and like compounds, which have attracted much attention as a remedy medicine for osteoporosis.

### BACKGROUND OF THE INVENTION

Zoanthamine alkaloids (cf. non-patent documents 1 to 11), which are heptacyclic alkaloids isolated from colonial zoanthids of Zoanthus sp., have attracted much attention from a wide area of science including medical chemistry, pharmacology, natural product chemistry, and synthetic organic chemistry because of their unique biological and pharmacological properties besides their novel chemical structure having stereochemical complexity. For example, norzoanthamine, which was isolated from zoanthids captured in the sea near the Amamiooshima island in Japan and structurally identified by UEMURA et. al. in 1995, is able to significantly suppress loss of bone weight and strength in ovariectomized mice and has been expected as a promising candidate as a remedy medicine for osteoporosis (ef. non-patent documents 3 and 12).

The following is a structural formula of norzoanthamine: 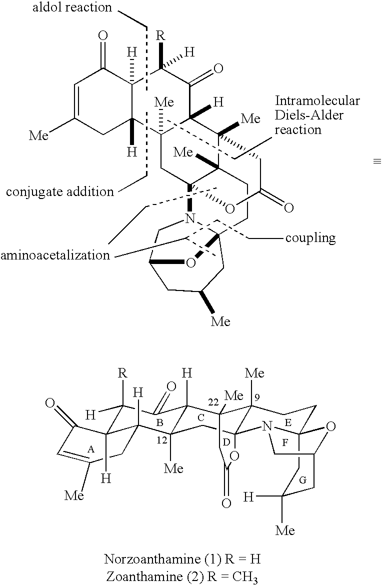
where R═H for norzoanthamine. On the other hand, zoanthamine isolated by Faulkner et. al. (which has the same structural formula as norzoanthamine except that R═CH₃ for zoanthamine) (cf. non-patent documents 4 and 5) has exhibited potent inhibitory activity toward phorbol myristate-induced inflammation in addition to powerful analgesic effects. Very recently, a norzoanthamine derivative was demonstrated to strongly inhibit growth of P-388 murine leukemia cell lines in addition to its anti-platelet activity on human platelet aggregation (cf. non-patent document 13). Therefore, norzoanthamine has been of keen interest, particularly, in relation to development of a new type of remedy medicine for osteoporosis for the advanced age (cf. non-patent documents 3 and 12). These unique biological properties combined with novel chemical structures make this family of alkaloids extremely attractive targets for chemical synthesis.

In the following the documents referred above are listed:

Non-Patent Document 1

S. Fukuzawa, Y. Hayashi, D. Uemura, A. Nagatsu, K. Yamada, Y. Ijuin, Heterocycl. Commun. 1, 207 (1995).

Non-Patent Document 2

M. Kuramoto, K. Hayashi, Y. Fujitani, K. Yamaguchi, T. Tsuji, K. Yamada, Y. Ijuin, D. Uemura, Tetrahedron Lett. 38, 5683 (1997).

Non-Patent Document 3

M. Kuramoto, K. Hayashi, K. Yamaguchi, M. Yada, T. Tsuji, D. Uemura, Bull. Chem. Soc. Jpn. 71, 771 (1998).

Non-Patent Document 4

C. B. Rao, A. S. R. Anjaneyula, N. S. Sarma, Y. Venkatateswarlu, R. M. Rosser, D. J. Faulkner, M. H. M. Chen, J. Clardy, J. Am. Chem. Soc. 106, 7983 (1984).

Non-Patent Document 5

C. B. Rao, A. S. R. Anjaneyula, N. S. Sarma, Y. Venkatateswarlu, R. M. Rosser, D. J. Faulkner, J. Org. Chem. 50, 3757 (1985).

Non-Patent Document 6

C. B. Rao, D. V. Rao, V. S. N. Raju, Heterocycles, 28, 103 (1989).

Non-Patent Document 7

A. Rahman, K. A. Alvi, S. A. Abbas, M. I. Choudhary, J. Clardy, Tetrahedron Lett. 30, 6825 (1989).

Non-Patent Document 8

A. H. Daranas, J. J. Fernandez, J. A. Gavin, M. Norte, Tetrahedron, 54, 7891 (1998).

Non-Patent Document 9

H. Nakamura, Y. Kawase, K. Maruyama, A. Murai, Bull. Chem. Soc. Jpn. 71, 781 (1998).

Non-Patent Document 10

Y. Venkateswarlu, N. S. Reddy, P. Ramesh, P. S. Reddy, K. Jamil, Heterocycl. Commun. 4, 575 (1998).

Non-Patent Document 11

A. H. Daranas, J. J. Fernandez, J. A. Gavin, M. Norte, Tetrahedron, 55, 5539 (1999).

Non-Patent Document 12

K. Yamaguchi, M. Yada, T. Tsuji, M. Kuramoto, D. Uemura, Biol. Pharm. Bull. 22, 920 (1999).

Non-Patent Document 13

R. M. Villar, J. G-Longo, A. H. Daranas, M. L. Souto, J. J. Fernandez, S. Peixinho, M. A. Barral, G. Santafe, J. Rodriguez, C. Jimenez, Bioorg. Med. Chem. 11, 2301 (2003).

Despite of intensive works to synthesize this compound (cf. non-patent documents 14 to 28), its densely functionalized complex stereostructure has prevented the alkaloid from being chemically synthesized.

In the following the documents referred above are listed:

Non-Patent Document 14

D. Tanner, P. G. Andersson, L. Tedenborg, P. Somfai, Tetrahedron, 50, 9135 (1994).

Non-Patent Document 15

D. Tanner, L. Tedenborg, P. Somfai, Acta. Chem. Scand. 51, 1217 (1997).

Non-Patent Document 16

T. E. Nielsen, D. Tanner, J. Org. Chem. 67, 6366 (2002).

Non-Patent Document 17

D. R. Williams, G. S. Cortez, Tetrahedron Lett. 39, 2675 (1998).

Non-Patent Document 18

D. R. Williams, T. A. Brugel, Org. Lett. 2, 1023 (2000).

Non-Patent Document 19

S. Ghosh, F. Rivas, D. Fisher, M. A. Gonzalez, E. A. Theodorakis, Org. Lett. 6, 941 (2004).

Non-Patent Document 20

G. Hirai, H. Oguri, M. Hirama, Chem. Lett. 141 (1999).

Non-Patent Document 21

S. M. Moharram, G. Hirai, K. Koyama, H. Oguri, M. Hirama, Tetrahedron. Lett. 41, 6669 (2000).

Non-Patent Document 22

G. Hirai, H. Oguri, S. M. Moharram, K. Koyama, M. Hirama, Tetrahedron. Lett. 42, 5783 (2001).

Non-Patent Document 23

G. Hirai, Y. Koizumi, S. M. Moharram, H. Oguri, M. Hirama, Org. Lett. 4, 1627 (2002).

Non-Patent Document 24

N. Hikage, H. Furukawa, K. Takao, S. Kobayashi, Tetrahedron. Lett. 39, 6237 (1998).

Non-Patent Document 25

N. Hikage, H. Furukawa, K. Takao, S. Kobayashi, Tetrahedron. Lett. 39, 6241 (1998).

Non-Patent Document 26

N. Hikage, H. Furukawa, K. Takao, S. Kobayashi, Chem. Pharm. Bull. 48, 137.0 (2000).

Non-Patent Document 27

M. Sakai, M. Sasaki, K. Tanino, M. Miyashita, Tetrahedron Lett. 43, 1705 (2002).

Non-Patent Document 28

Digest of Speeches at the 45th Natural Organic Chemicals Discussion, pp.121-125, 2003.

### SUMMARY OF THE INVENTION

An object of the present invention is to provide a method of producing a zoanthamine alkaloid such as norzoanthamine and the like compounds with high yield, and an intermediate suitable for use in the method.

As a result of diligent works, the inventors of the present invention found out a most suitable synthetic route for a zoanthamine alkaloid such as norzoanthamine and the like compounds. The present invention is based on this finding. Further, the inventor of the present invention found out that the following strategy is effective for the synthesis of the zoanthamine alkaloid such as norzoanthamine and the like compounds.

(1) C ring, which is so stereochemically dense that it has three adjacent quaternary asymmetric carbon atoms at C-9, C-12, and C-22 positions, is constructed by a thermal reaction (intramolecular Diels-Alder Reaction) of triene;

(2) stereoselective synthesis of requisite triene, which is a key intermediate and a precursor of the Diels-Alder Reaction, is carried out by three-component coupling reactions, which involve a conjugate addition of a vinyl cupurate reagent to (R)-5-methyl-2-cyclohexenone, followed by an aldol reaction and subsequent photosensitized oxidation of a furan ring;

(3) an amino-alcohol side chain is constructed from citroneral, which is commercially available;

(4) an unprecedented synthetic route using deuterium is designed to attain efficient synthesis of a crucial alkyne derivative, which is a key three-ring compound;

(5) regioselective introduction of a double bond into an A ring should be performed before aminoacetalization, in order that the double bond may be introduced into the A ring with high efficiency;

(6) the aminoacetalization to form two aminoacetal structures is carried out including initial treatment with aqueous acetic acid followed by treatment with aqueous trifluoroacetic acid under reflux, so that the aminoacetal structures, which are unstable, can be synthesized under mild conditions.

This strategy to solve the problems associated with the synthesis of the zoanthamine alkaloids is applicable to synthesis of various kinds of zoanthamine-based alkaloids such as norzoanthamine, zoanthamine, and the like compounds.

In order to solve the aforementioned problem, a first arrangement of the present invention is a method of producing a zoanthamine alkaloid including the steps of converting a first compound to a second compound; converting the second compound to a third compound; converting the third compound to a fourth compound; converting the fourth compound to a fifth compound; converting the fifth compound to a sixth compound; and converting the sixth compound to the zoanthamine alkaloid, the first compound represented by: 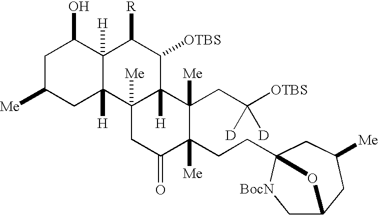
the second compound represented by: 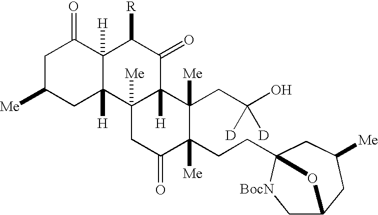
the third compound represented by: 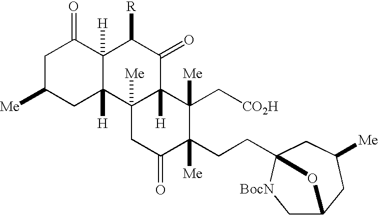
the fourth compound represented by: 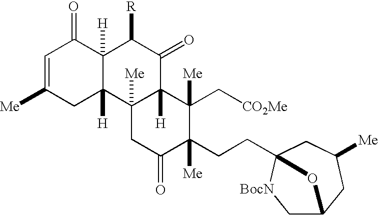
the fifth compound represented by: 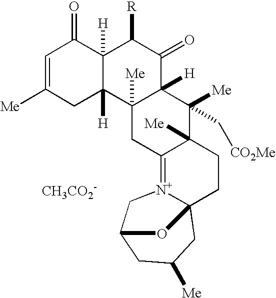
the sixth compound represented by: 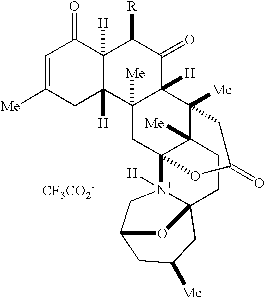
where R is H or CH₃, D is a deuterium, TBS is a tert-butyldimethylsilyl group, Boc is a tert-butoxycarbonyl group, and Me is a methyl group.

A second arrangement of the present invention is a method of producing a zoanthamine alkaloid, including the steps of: removing two tert-butyldimethylsilyl groups from a first compound and selectively oxidizing secondary hydroxyl groups of the first compound, so as to obtain a second compound; oxidizing the second compound to an aldehyde and oxidizing the aldehyde to a carboxylic acid, so as to obtain a third compound; esterifying the third compound and introducing a double bond into an A-ring of the esterificated third compound, so as to obtain a fourth compound; producing an iminium salt of the fourth compound, so as to obtain a fifth compound; producing an ammonium salt of the fifth compound, so as to obtain a sixth compound; and desalinating the sixth compound, so as to obtain the zoanthamine alkaloid, the first compound represented by: 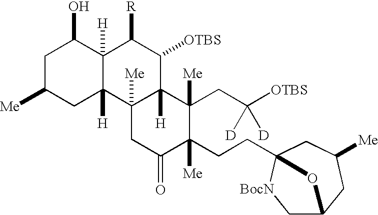
the second compound represented by: 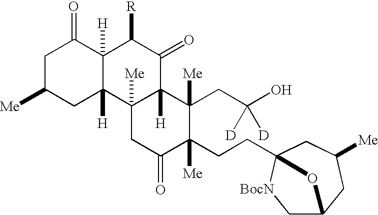
the third compound represented by: 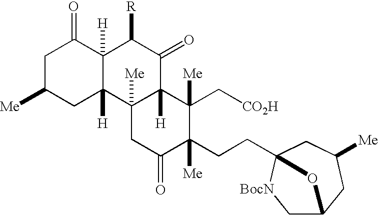
the fourth compound represented by: 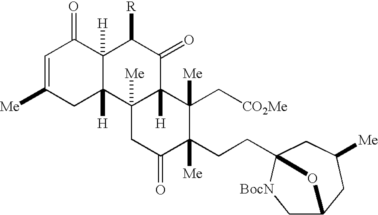
the fifth compound represented by: 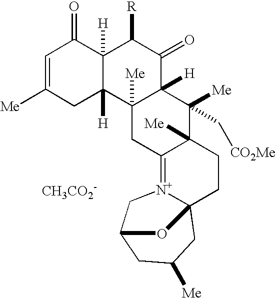
the sixth compound represented by: 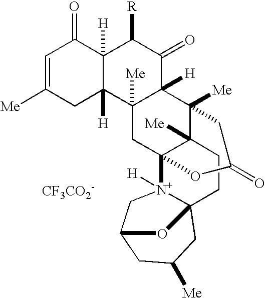
where R is H or CH₃, D is a deuterium, TBS is a tert-butyldimethylsilyl group, Boc is a tert-butoxycarbonyl group, and Me is a methyl group.

A third arrangement of the present invention is a method of producing a zoanthamine alkaloid, including the steps of: converting a seventh compound to an eighth compound; converting the eighth compound to a ninth compound; converting the ninth compound to a tenth compound; converting the tenth compound to an eleventh compound; converting the eleventh compound to a twelfth compound; and converting the twelfth compound to a thirteenth compound, the seventh compound represented by: 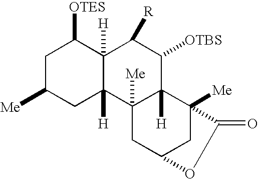
the eighth compound represented by: 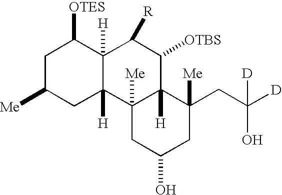
the ninth compound represented by: 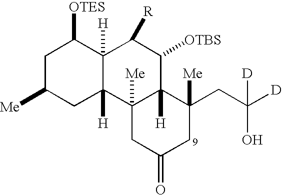
the tenth compound represented by: 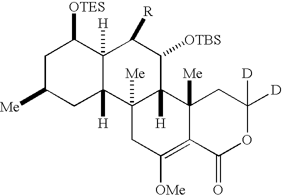
the eleventh compound represented by: 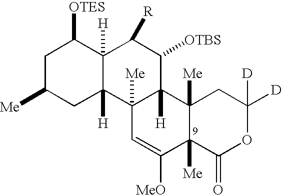
the twelfth compound represented by: 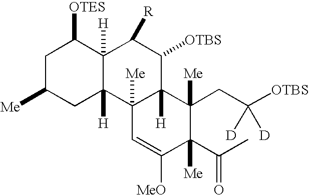
the thirteenth compound represented by: 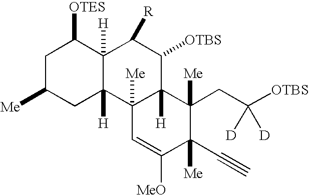
where R is H or CH₃, D is a deuterium, TBS is a tert-butyldimethylsilyl group, TES is a triethylsilyl group, and Me is a methyl group.

A fourth arrangement of the present invention is a method of producing a zoanthamine alkaloid, including the steps of: subjecting a seventh compound to (a) reduction, (b) a Wittig reaction with a compound containing a deuterium, and (c) hydroboration, so as to obtain an eighth compound; subjecting the eighth compound to oxidation so as to obtain a ninth compound; forming a carbonate of the ninth compound, and subjecting the carbonate of the ninth compound to an intramolecular acylation reaction and subsequent methylation reaction, so as to obtain a tenth compound; introducing a methyl group at a C-9 position of the tenth compound so as to obtain an eleventh compound; adding a methyl group to a carbon that is bound with an oxygen with which deuteriums are bound, so as to obtain a twelfth compound; and converting a methyl ketone of the twelfth compound to a triple bond, so as to obtains a thirteen compound,

the seventh compound represented by: 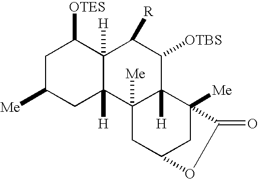
the eighth compound represented by: 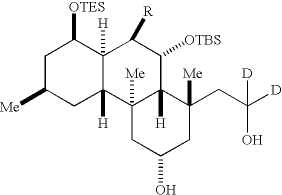
the ninth compound represented by: 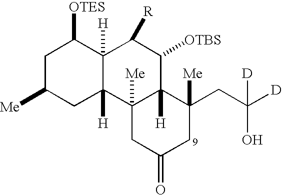
the tenth compound represented by: 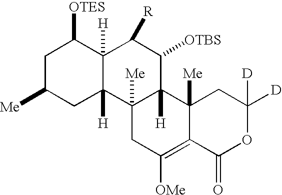
the eleventh compound represented by: 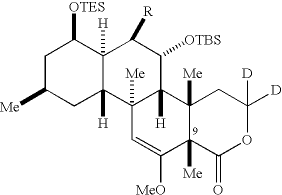
the twelfth compound represented by: 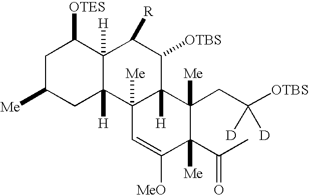
the thirteenth compound represented by: 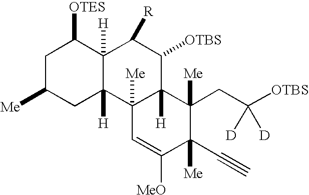
where R is H or CH₃, D is a deuterium, TBS is a tert-butyldimethylsilyl group, TES is a triethylsilyl group, and Me is a methyl group.

A fifth arrangement of the present invention is an intermediate represented by: 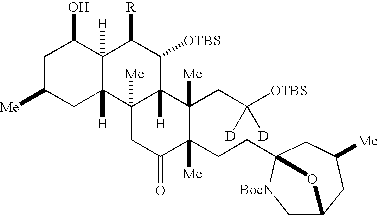
where R is H or CH₃, TBS is a tert-butyldimethylsilyl group, Boc is a tert-butoxycarbonyl group, and Me is a methyl group.

A sixth arrangement of the present invention is an intermediate represented by: 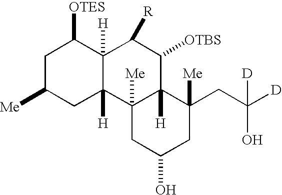
where R is H or CH₃, D is a deuterium, TBS is a tert-butyldimethylsilyl group, TES is a triethylsilyl group, and Me is a methyl group.

A seventh arrangement of the present invention is an intermediate represented by: 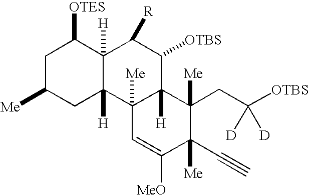
where R is H or CH₃, D is a deuterium, TBS is a tert-butyldimethylsilyl group, TES is a triethylsilyl group, and Me is a methyl group.

According to the first and second arrangements, it is possible to produce a zoanthamine alkaloid with a high yield, through suitable synthetic routes via the second to sixth compounds starting from the first compound.

According to the third to fourth arrangements, it is possible to effectively suppress formation of a by-product in synthesizing the thirteen compound, because the thirteen compound is formed by the synthetic reaction starting from the eighth compound, which is synthesized by deuterium substitution of the seventh compound. As a result, it becomes possible to produce a zoanthamine alkaloid with a high yield.

According to the fifth to seventh arrangements, it is possible to produce a zoanthamine alkaloid with high yield, via a suitable synthetic route.

### BRIEF DESCRIPTION OF THE DRAWINGS

FIG. 1 is a scheme illustrating a method for producing norzoanthamine according to an exemplary embodiment of the present invention.

FIG. 2 is a scheme illustrating the method for producing norzoanthamine according to the exemplary embodiment of the present invention.

FIG. 3 is a scheme illustrating the method for producing norzoanthamine according to the exemplary embodiment of the present invention.

FIG. 4 is an NMR spectrum of norzoanthamine produced by the method for producing norzoanthamine according to the exemplary embodiment.

FIG. 5 is an NMR spectrum of naturally-occurring norzoanthamine.

### DESCRIPTION OF THE EMBODIMENT

A method of producing norzoanthamine according to an exemplary embodiment of the present invention is described below.

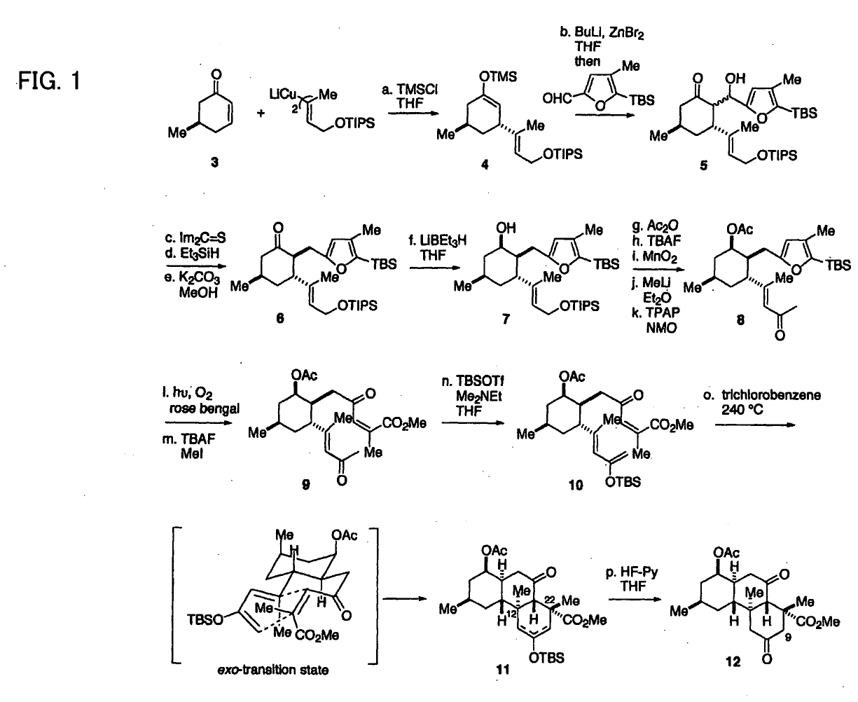

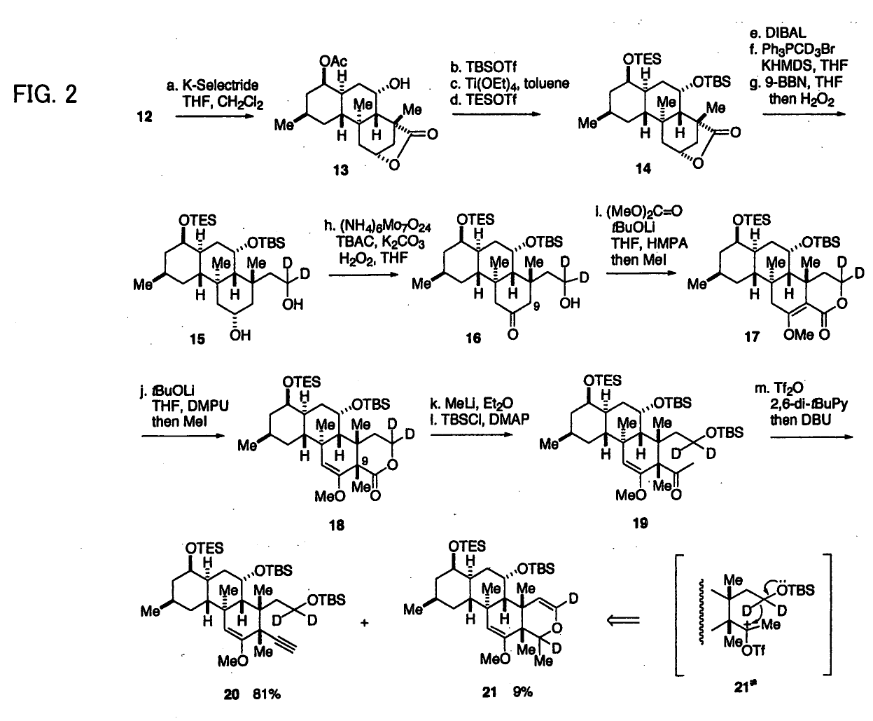

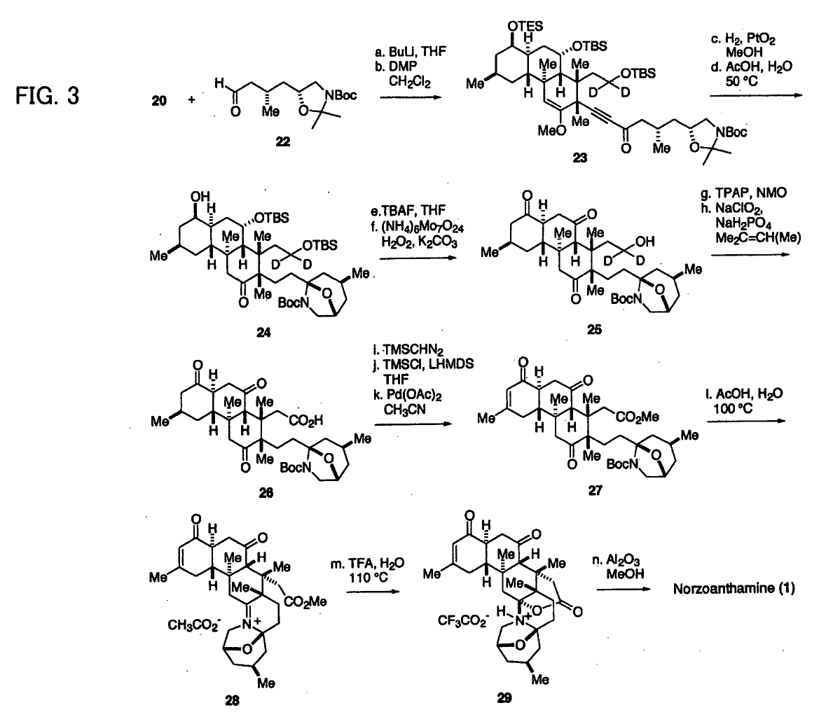

FIGS. 1, 2, and 3 illustrate the whole steps of the method of producing norzoanthamine according to the exemplary embodiment. FIG. 1 illustrates a method of synthesizing an ABC ring system. FIG. 2 illustrates a method of synthesizing an alkyne segment using deuterium. FIG. 3 illustrates the entire method of synthesizing norzoanthamine.

The following describes the method of producing norzoanthamine stepwise.

Firstly, the ABC ring of norzoanthamine is synthesized as follows.

The synthesis of the ABC ring is started from (R)-5-methyl-2-cyclohexenone (cf. S. Mutti, C. Daubie, F. Decalogne, R. Fournier, P. Rossi, Tetrahedron. Lett. 43, 1705 (2002)), whose structural formula is as follows:

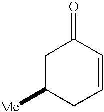

In the presence of chlorotrimethylsilane (TMSCI) (cf. E. Nakamura, S. Matsuzawa, Y. Horiguchi, I. Kuwajima, Tetrahedron. Lett. 27, 4029 (1986)), a conjugate addition of lithium (E)-di[4-triisopropylsilyloxy]-2-butenylcupurate to (R)-5-methyl-2-cyclohexenone is carried out to obtain silyl enol ether stereoselectively. This conjugate addition may be carried out, e.g., at −40° C. for one hour. The lithium (E)-di[4-triisopropylsilyloxy]-2-butenylcupurate used here has the following structural formula: 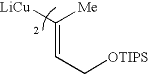
silyl enol ether thus obtained has the following structural formula:

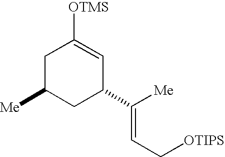

Then, silyl enol ether thus obtained is subjected to an aldol reaction with functionalized furaldehyde via a zinc enolate.

More specifically, the silyl enol ether thus obtained may be reacted with butyllithium (BuLi) in THF, then with zinc bromide (ZnBr₂), and further with 4-methyl-5-[tert-butyldimethylsilyl]frufural. The reaction with butyllithium (BuLi) is carried out, e.g., at −30° C. for two hours. The reaction with zinc bromide (ZnBr₂) may be carried out, e.g., at −78° C. for two hours. The reaction with 4-methyl-5-[tert-butyldimethylsilyl]frufural may be carried out, e.g., at −78° C. for three hours. 4-Methyl-5-[tert-butyldimethylsilyl]frufural has the following structural formula: 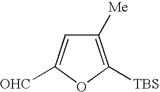
This aldol reaction gives an aldol as a diastereoisomeric mixture. A structural formula of the aldol is as follows: 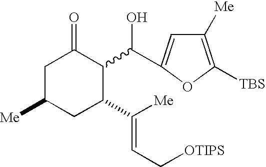
In an example in which this aldol reaction was actually performed by way of trial, the aldol reaction had a yield of 84% (for two steps). In the aldol reaction, conjugate addition of vinylcupurate occurs from an opposite side of a secondary methyl group on a cyclohexene ring.

Results of physical analysis of resultant compounds were as follows.

Compound (β): [α]²⁸_{D}=−6.6° (c 1.50, CHCl₃); IR (neat) 3450, 2866, 1703, 1464, 1250, 883, 775 cm⁻¹; ¹H NMR (CDCl₃, 270 MHz)δ 0.23 (s,3 H), 0.24 (s,3 H), 0.88 (s, 9 H), 0.91 (d, J=6.8 Hz, 3 H), 1.02-1.10 (m, 21 H, involving a singlet at 1.07), 1.50-1.65 (m, 4 H, involving a singlet at 1.57), 1.90 (ddd,J=4.6, 11.7, 13.7 Hz, 1 H), 2.05 (s, 3 H), 2.16-2.38(m, 2 H), 2.46 (dd,J=4.5, 13.7 Hz, 1 H), 2.59 (dt, J=4.6, 11.2 Hz, 1 H), 2.83 (dd,J=4.3, 11.2 Hz, 1 H), 4.12-4.30 (m, 3 H, involving a doublet at 4.15, J=10.2 Hz), 4.66 (dd, J=4.5, 10.2 Hz, 1 H), 5.36 (bt,J=5.6 Hz, 1 H), 6.08 (s, 1 H); ¹³C NMR (67.8 MHz, CDCl₃)δ −5.56,−5.53, 11.59, 12.10 (3 C), 13.15, 17.89, 18.12 (6 C), 20.18, 26.54 (3 C), 28.45, 35.73, 43.14 , 48.21, 55.29, 60.25, 67.77, 111.55, 127.99, 131.96, 135.11, 152.15, 158.14, 214.20; HRMS Calcd for C₃₂H₅₈O₄Si₂ ([M]⁺); 562.3874. Found: 562.3877.

Compound (α):[α]²⁸_{D}=+80.6° (c 0.64, CHCl₃); IR (neat) 3500, 2866, 1705, 1464, 1250, 1103, 1061, 883 cm⁻¹; ¹H NMR (CDCl₃, 270 MHz)δ 0.22 (s, 6 H), 0.86 (s, 9 H), 1.00-1.12 (m, 24 H, involving a singlet at 1.06 and a doublet at 1.00, J=6.9 Hz), 1.60-1.68 (m, 4 H, involving a singlet at 1.62), 1.95-2.15 (m, 5 H, involving a singlet at 2.05), 1.95-2.15 (m, 5 H, involving a singlet at 2.05), 2.35-2.48 (m, 1 H), 2.57 (dd, J=5.6, 12.9 Hz, 1 H), 2.89 (dt, J=4.0, 10.9 Hz, 1 H), 2.98 (dd, J=2.5, 10.9 Hz, 1 H), 3.77 (d, J=10.9 Hz, 1 H), 4.27 (bd, J=5.8 Hz, 2 H), 4.63 (dd, J=2.0, 10.9 Hz, 1 H), 5.57 (bt, J=5.8 Hz, 1 H), 6.10 (s, 1 H); ¹³C NMR(67.8 MHz, CDCl₃)δ −5.67, −5.63, 11.54, 12.12 (3 C), 13.25, 17.80, 18.10 (6 C), 19.21, 26.40 (3 C), 30.80, 36.15, 45.75, 48.89, 55.42, 60.28, 67.25, 109.16, 128.32, 132.26, 135.56, 151.54, 159.74, 213.94; HRMS Calcd for C₃₂H₅₈O₄Si₂ ([M]⁺);562.3874.Found: 562.3849.

Next, with 1,1′-thiocarbonyldiimidazol (Im₂C═S), the aldol thus obtained is dehydrated, thereby obtaining enones (E/Z=96:4). More specifically, the dehydration is carried out, e.g., at 70 to 90° C. for 6.5 hours. In the example in which this dehydration was actually performed by way of trial, the dehydration had a 92% yield.

Next, in the presence of a Wilkinson catalyst, enones is subjected to a hydrosilylation reaction (I. Ojima, T. Kogure, Organometallics. 1, 1390(1982)). The hydrosilylation reaction gives silyl enol ether. More specifically, the hydrosilylation reaction may be carried out, e.g., with triethylsilane (Et₃SiH) in THF in the presence of chlorotris(triphenylphosphine)rhodium(I) as the Wilkinson catalyst. The hydrosilylation reaction is carried out, e.g., at 50° C. for two hours.

Next, silyl enol ether obtained from the hydrosilylation reaction is treated with K₂CO₃ in THF and methanol. This treatment of silyl enol ether with K₂CO₃ may be carried out, e.g., at room temperature for one hour. In the example in which this treatment was actually carried out by way of trial, this treatment gave a 86% yield (for two steps). Overall, a trisubstituted cyclohexanone is obtained with a desired stereochemistry through these reactions and treatment. The trisubstituted cyclohexanone has the following structural formula: 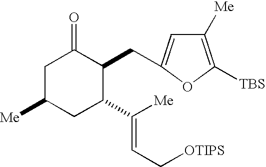
In the example in which these reactions and treatment were carried out by way of trial, the overall yield (i.e. a yield of trisubstituted cyclohexanone) was 79% (for three steps).

Results of physical analysis of the trisubstituted cyclohexanone were as follows:

[α]³⁰_{D}=+23.0° (c 4.00, CHCl₃); IR (neat) 2866, 1713, 1464, 1389, 1250, 1111, 1061, 883, 758 cm⁻¹; ¹H NMR (CDCl₃, 270 MHz)δ 0.21 (s, 6 H), 0.87 (s, 9 H), 0.95 (d, J=7.1 Hz, 3 H), 1.02-1.14 (m, 21 H, involving a singlet at 1.07), 1.52-1.62 (m, 4 H, involving a broad singlet at 1.57), 1.93-2.04 (m, 4 H, involving a singlet at 2.01), 2.13-2.19 (m, 1 H), 2.33-2.57 (m, 4 H), 2.68-2.77 (m, 1 H), 2.97 (dd, J=8.2, 15.0 Hz, 1 H), 4.27 (bd, J=5.8 Hz, 2 H), 5.42(bt, J=5.8 Hz, 1 H), 5.82 (s, 1 H); ¹³C NMR (67.8 MHz, CDCl₃)δ −5.58, −5.55, 11.54, 12.12 (3 C),12.67, 17.81, 18.10 (6 C), 19.42, 25.64, 26.47 (3 C), 30.37, 36.22, 48.03, 48.63, 51.93, 60.32, 109.61, 127.93, 132.15, 135.98, 150.72, 157.82, 210.77; HRMS Calcd for C₃₂H₅₈O₃Si₂ ([M]⁺); 546.3924. Found: 546.3934.

Next, the trisubstituted cyclohexanone was reduced with lithium triethylborohydride (LiBEt₃H) in THF. The reduction is carried out, e.g., at −78° C. for 30 minutes. As a result, a single β-alcohol is obtained nearly purely. The β-alcohol has the following structural formula: 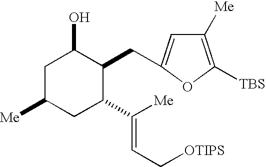
In the example in which the reduction was actually performed by way of trial, a yield of the β-alcohol was 98%.

Results of physical analysis of the β-alcohol were as follows:

[α]³⁰_{D}=+6.7° (c 1.50, CHCl₃); IR (neat) 3450, 2866, 1603, 1464, 1389, 1250, 1101, 1059, 883, 835 cm⁻¹; ¹H NMR (CDCl₃, 270 MHz)δ 0.23 (s, 3 H),0.23 (s, 3 H), 0.88 (s, 9 H), 1.02-1.11 (m, 21 H, involving a singlet at 1.07), 1.13 (d,J=7.3 Hz, 3 H), 1.35-1.43 (m, 1 H), 1.51-1.72 (m, 8 H, involving a singlet at 1.54), 1.80-2.00 (m, 2 H), 2.04 (s, 3 H), 2.37 (bdt, J=3.6, 9.6 Hz, 1 H), 2.59 (dd, J=2.0, 7.8 Hz, 1 H), 3.76-3.82 (m, 1 H), 4.29 (bd, J=5.8 Hz, 2 H), 5.45 (bt, J=5.8 Hz, 1 H), 5.83 (s, 1 H); ¹³C NMR (67.8 MHz, CDCl₃) δ −5.61, −5.56, 11,57, 12,17 (3 C), 13.70, 17.84, 18.12 (6 C), 21.56, 26.47 (3 C), 27.07, 27.56, 35.98, 37.95, 41.44, 43.07, 60.58, 68.29, 109.59, 126.29, 132.13, 137.65, 151.39, 158.52; HRMS Calcd for C₃₂H₆₀O₃Si₂ ([M]⁺); 548.4081. Found: 548.4113.

Next, the β-alcohol is converted to a methyl ketone by a routine five-step reaction sequence: (i) acetylation, (ii) removal of triisopropylsilyl (TIPS) group, (iii) oxidation with manganese dioxide (MnO₂), (iv) addition of methyllithium (MeLi) to aldehyde, and (v) oxidation of a secondary alcohol with tetrapropylammonium perruthenate (TPAP). The methyl ketone has the following structural formula: 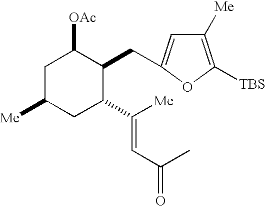
In the following a more specific example of the routine five-step reaction sequence is described. The reaction (i) may be carried out with acetic anhydride (Ac₂O), pyridine, 4-dimethylamiopyridine, dichloromethane (CH₂Cl₂), e.g., at room temperature for 1.5 hours. The reaction (ii) may be carried out with tetra-n-butylammonium fluoride (TBAF) in THF, e.g., at room temperature for 5.5 hours. In the example in which the reaction (ii) was actually carried out by way of trial, a yield of the reaction (ii) was 96% (for two steps). The reaction (iii) may be carried out with MnO₂ in CH₂Cl₂, e.g., at room temperature for thirteen hours. The reaction (iv) may be carried out with MeLi in ether (Et₂O), e.g., at −100° C. for three hours. The reaction (v) may be carried out with TPAP, and 4-methylmorpholine-N-oxide (NMO) in CH₂Cl₂ by using a molecular sieve 4A, e.g., at room temperature for 1.5 hours. In the example in which the reaction (v) was actually carried out by way of trial, a yield of the reaction (v) was 95% (for three steps). An overall yield of the routine five-step reaction sequence was 91% in the example in which the routine five-step reaction sequence was actually carried out by way of trial.

Results of physical analysis of the methyl ketone obtained via the routine five-step reaction sequence were as follows:

[α]³⁰^{D}=−26.2° (c 1.63, CHCl₃); IR (neat) 2928, 2856, 1740, 1688, 1611, 1244, 775 cm⁻¹; ¹H NMR (CDCl₃, 270 MHz)δ 0.22 (s, 3 H), 0.22 (s, 3 H), 0.87 (s,9 H), 1.06 (d, J=7.1 Hz, 3 H), 1.82 (m, 4 H), 1.92-2.21 (m, 14 H, involving singlets at 2.02, 2.07, 2.21 and a doublet at 2.09, J=1.2 Hz), 2.41-2.53 (m, 3 H), 4.92 (dt, J=3.3, 5.1 Hz, 1 H), 5.76 (s, 1 H), 6.26. (s, 1 H); ¹³C NMR (67.8 MHz, CDCl₃)δ −5.63, −5.60, 11.51, 16.46, 17.80, 21.03, 21.39, 26.45 (3 C), 26.65, 27.53, 32.04, 34.50, 35.53, 40.64, 43.89, 70.94, 109.91, 124.45, 132.03, 151.67, 156.73, 159.91, 170.06, 198.55; HRMS Calcd for C₂₂H₃₃O₄Si([M-tDu]⁺);.389.2148. Found: 389.2176.

Next, photosensitized oxidation of a furan ring is performed according to the Katsumura protocol with a halogen lamp and rose bengal (cf. S. Katsumura, K. Hori, S. Fujiwara, S. Isoe, Tetrahedron. Lett. 39, 4625 (1985)). The photosensitized oxidation gives a Z-γ-keto-α,β-unsaturated silyl ester in quantitative yield. More specifically, the photosensitized oxidation of the furan ring may be carried out with light radiation by using a halogen lamp under oxygen in the presence of rose bengal in CH₂Cl₂. The photosensitized oxidation may be carried out, e.g., at 0° C. for 12 hours. Z-γ-keto-α,β-unsaturated silyl ester is immediately converted to a stable methyl ester using tetrabutylammonium fluoride (TBAF) and iodomethane in THF (T. Ooi, H. Sugimoto, K. Maruoka, Heterocycles, 54, 593 (2001)). The methyl ester has the following structural formula: 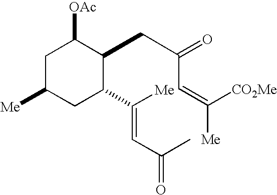
The conversion of Z-γ-keto-α,β-unsaturated silyl ester to the methyl ester may be carried out, e.g., at room temperature for one hour. In the example in which this conversion was actually performed by way of trial, a yield of the conversion was 97% (for two steps).

Results of physical analysis of the methyl ester were as follows:

[α]²⁸_{D}=−30.0° (c 1.35, CHCl₃); IR (neat) 2953, 1736, 1688, 1612, 1371, 1246, 1138, 1028, 964 cm⁻¹; ¹H NMR (CDCl₃, 270 MHz)δ 1.05 (d, J=7.1 Hz, 3 H), 1.43 (dt, J=4.5, 13.4 Hz, 1 H), 1.63-1.82 (m, 3 H), 1.92-2.58 (m, 17 H, involving singlets at 2.04, 2.21 and doublets at 2.00, J=1.6 Hz, 2.06, J=1.2 Hz), 3.77(s, 3 H), 5.04-5.08 (m, 1 H), 6.08 (q, J=1.6 Hz, 1 H), 6.22 (s, 1 H); ¹³C NMR (67.8 MHz, CDCl₃) δ 16.96, 20.28, 21.02, 21.37, 26.52, 32.00, 34.62, 35.29, 36.12, 42.10, 43.97, 52.33, 71.47, 124.69, 129.89, 141.09, 159.13, 169.02, 170.10, 197.97, 198.54; HRMS Calcd for C₂₁H₃₀O₆ ([M]⁺); 378.2042. Found: 378.2060.

After the photosensitized oxidation, the resultant methyl ester was treated with tert-butyldimethylsilyl trifluoromethanesulfonate (TBSOTf) and N,N-dimethylethylamine (Me₂NEt) in THF, thereby obtaining triene. This treatment is carried out, e.g., at 0° C. for 30 minutes. Triene has the following structural formula: 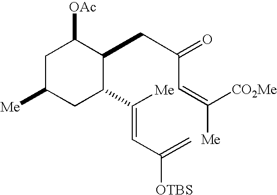
In the example in which this treatment was actually carried out by way of trial, a yield of the triene was 100%.

Results of physical analysis of the resultant triene were as follows:

[α]²⁸_{D}=−9.7° (c 1.13, CHCl₃); IR (neat) 2930, 2858, 1738, 1369, 1246, 1136, 837, 781 cm⁻¹; ¹H NMR (CDCl₃, 270 MHz)δ 0.17 (s, 6 H), 0.94 (s, 9 H), 1.05(d, J=7.3 Hz, 3 H), 1.37-1.46 (m, 1 H), 1.67-1.80 (m, 5 H, involving a doublet at 1.80, J=1.2 Hz), 1.97-2.07 (m, 8 H, involving a singlet at 2.04 and a doubletat 1.99, J=1.6 Hz), 2.27-2.44 (m, 4 H), 3.76 (s, 3 H), 4,21 (s, 1 H), 4.35(s, 1 H), 5.02-5.06 (m, 1 H), 5.68 (s, 1 H), 6.08 (q, J=1.6 Hz, 1 H); ¹³C NMR(67.8 MHz, CDCl₃)δ −4.27 (2 C), 15.03, 18.35, 20.30, 20.95, 21.46, 25.91 (3 C), 26.56, 34.69, 35.53, 36.98, 42.40, 43.09, 52.30, 72.13, 95.92, 124.72, 130.28, 140.62, 141.16, 155.14, 169.17, 170.21, 198.56; HRMS Calcd for C₂₇H₄₄O₆Si ([M]⁺); 492.2907.Found:492.2878.

A next step is an intramolecular Diels-Alder reaction, which is critically important. The Diels-Alder reaction is carried out by adding dropwise a solution in which the triene is dissolved in 1,2,4-trichlorobenzene, into 1,2,4-trichlorobenzene heated to, e.g., 240° C. The Diels-Alder reaction is continued, e.g., for 1.5 hours at 240° C. The Diels-Alder reaction, which proceeds smoothly, gives exo and endo adducts via an exo transition state (which is described later). In the example in which the Diels-Alder reaction was actually carried out, the exo and endo adducts were obtained as a 72:28 mixture with 98% combined yield. The following is a structural formula of the exo-transition state: 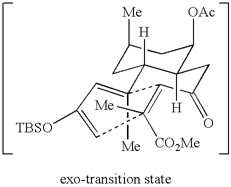
The following is a structural formula of the exo adducts:

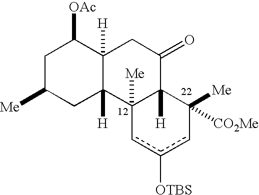

Next, the resultant adducts are treated with hydrogen fluoride (HF)-pyridine in THF, whereby causing a simple crystallization gives a crystalline compound. This treatment is carried out, e.g., at room temperature for three hours. The crystalline compound has the following structural formula: 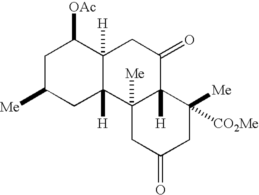
In the example in which this treatment (i.e. the crystallization) was carried out, a yield of the crystalline compound was 51% (for two steps). The crystalline compound obtained in the example was analyzed by X-ray crystallographic analysis to find a stereostructure of the crystalline compound. The X-ray crystallographic analysis unambiguously confirmed that the stereostructure of the crystalline compound was that of the exo adduct. This analysis showed that the intramolecular Diels-Alder reaction of the triene occurred stereoselectively via the exo transition state to give rise to an ABC ring system with two quaternary asymmetric carbon centers at the C-12 and C-22 positions. The process up to this stage is 16 steps. In the example in which the process up to this state was actually carried out, a total yield of the crystalline compound from 5-methylcyclohexenone was remarkably high, 29%.

Results of physical analysis of the crystalline compound (exo adduct) were as follows:

mp 203-204° C.;[α]³⁰_{D}=−73.1° (c 1.70, CHCl₃); IR (CHCl₃) 3022, 2951, 1732, 1466, 1435, 1312, 1244, 1170, 1126, 1097, 1051, 1024, 943, 754 cm⁻¹; ¹H NMR(CDCl₃, 270 MHz) δ 1.04 (s, 3 H), 1.13 (d, J=7.4 Hz, 3 H), 1.35-1.46 (m, 4 H, involving a singlet at 1.39), 1.52-1.69 (m, 2 H), 1.88-2.35 (m, 11 H, involving a singlet at 2.11), 2.52 (dd, J=1.3, 14.7 Hz, 1 H), 2.72 (dd, J=1.3, 14.7 Hz, 1 H), 2.78 (s, 1 H), 3.70 (s, 3 H), 4.91 (q, J=3.0 Hz, 1 H); ¹³C NMR (67.8 MHz, CDCl₃)δ 16.09, 20.66, 21.44, 26.23, 28.63, 30.65, 34.45, 40.70, 43.33, 44.16, 45.11, 45.78, 50.54, 52.12, 52.52, 66.53, 72.47, 170.09, 174.97, 206.32, 206.76; HRMS Calcd for C₂₁H₃₀O₆ ([M]₊); 378.2042. Found: 378.2076. Stereoisomer (endo adducts): mp 230-233° C.; [α]³⁰_{D}=−82.7° (c 1.00, CHCl₃); IR(CHCl₃) 2878, 1724, 1366, 1240, 1097, 1022, 945 cm⁻¹; ¹H NMR (CDCl₃, 270 MHz)δ 1.13 (d, J=7.4 Hz, 3 H), 1.20 (dd, J=4.6, 12.7 Hz, 1 H), 1.28 (s, 3 H), 1.38 (s, 3H), 1.61-2.25 (m, 12 H, involving a singlet at 2.11 and a double doublet at 2.23, J=3.8, 11.7 Hz), 2.50 (d, J=14.7 Hz, 1 H), 2.59 (bt, J=12.6 Hz, 1 H), 3.18 (s , 1 H), 3.24 (d, J=14.7 Hz, 1 H), 3.65 (s, 3 H), 4.94-4.95 (m, 1 H); ¹³C NMR (67.8 MHz, CDCl₃) δ 20.20, 21.41, 26.39, 26.82, 27.96, 31.53, 34.92, 39.49, 43.17, 44.02, 44.77, 46.19, 47.08, 47.29, 52.26, 62.70, 73.28, 170.05, 177.36, 209.63, 211.11; HRMS Calcd for C₂₁H₃₀O₆ ([M]⁺); 378.2042. Found: 378.2044.

Next, construction of another quaternary asymmetric carbon center at the C-9 position is carried out. In order to construct this particular stereogenic center stereoselectively, a synthetic route as described below is designed. The synthetic route includes, as its key steps, an intramolecular acylation reaction of a keto alcohol and subsequent C-methylation reaction of a keto lactone, which is resulted from the intramolecular acylation reaction.

First, the crystalline compound was converted to hydroxy lactone in a highly stereoselective manner by treatment with potassium tri-sec-butylborohydride (K-Selectride) in THF and CH₂Cl₂. Hydroxy lactone has the following structural formula: 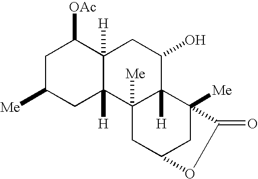
More specifically, for instance, this conversion of the crystalline compound to hydroxy lactone may be carried out, e.g., at −78° C. and then at −10° C. for 11 hours.

In the example in which reduction with K-selectride was actually performed by way of trial, a yield was 82%.

Next, the resultant hydroxy lactone is subjected to a three-step reaction sequence, thereby obtaining a compound (hereinafter, compound A) whose structural formula is as follows: 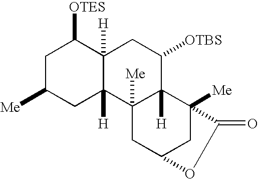
The three-step reaction sequence includes: (a) protection of a secondary alcohol with a tert-butyldimethylsilyl (TBS) group; (b) removal of acetate; and (c) protection of a hydroxyl group in the A-ring with a triethylsilyl (TES) group. A more specific example of the three-step reaction sequence is as follows: the step (a) may be carried out with TBSOTf and 2,6-lutidine (2,6-dimethylpyridine) in CH₂Cl₂, e.g., at room temperature for four hours; the step (b) may be carried out with titanium(IV) ethoxide (Ti(OEt)₄) in toluene, e.g., at 100° C. for 24 hours; the step (c) may be carried out with triethylsilyl trifluoromethanesulfonate (TESOTf) and 2,6-lutidine in CH₂Cl₂, e.g., at 0° C. for one hour. In the example in which the three-step reaction sequence was actually performed by way of trial, a yield was 90% (for three steps).

Results of physical analysis of the compound A were as follows:

[α]²⁶_{D}=−9.6° (c 1.25, CHCl₃); IR (CHCl₃) 2953, 1778, 1508, 1254, 1099 cm⁻¹; ¹H NMR (CDCl₃, 270 MHz)δ 0.05 (s, 3 H), 0.16 (s, 3 H), 0.59 (bq, J=8.1 Hz, 6 H), 0.91 (s, 9 H), 0.97 (bt, J=8.1 Hz, 9 H), 1.13 (d, J=7.4 Hz, 3 H), 1.23 (s, 3 H), 1.25 (s, 3 H), 1.28-1.69 (m, 10 H), 1.80 (d, J=11.2 Hz, 1 H), 1.95-2.00 (m, 1 H), 2.08 (ddd, J=1.6, 4.7, 13.9 Hz, 1 H), 2.26 (ddd, J=1.6, 5.9, 11.0 Hz, 1 H), 3.72-3.77 (m, 1 H), 4.32-4.37 (m, 1 H), 4.71 (bt, J=5.1 Hz, 1 H); ¹³C NMR (67.8 MHz, CDCl₃)δ −4.65, −3.71, 5.12 (3 C), 7.21 (3 C), 18.11, 18.70, 21.40, 21.45, 25.89 (3 C), 27.21, 31.17, 36.78, 37.75, 38.54, 38.58, 42.51, 43.66, 44.48, 49.34, 58.56, 67.88, 71.61, 75.49, 179.94, HRMS Calcd for C₂₆H₄₇O₄Si₂ ([M-tDu]⁺); 479.3013. Found: 479.3049.

Next, the compound A is reduced with diisobutylaluminum hydride (DIBAL) in toluene, thereby obtaining lactol. This reduction may be carried out, e.g., at −78° C. for two hours and repeated three times. The resultant lactol is subjected to a Wittig reaction with methyl-d3-triphenylphosphonium bromide (Ph₃PCD₃Br). This Wittig reaction gives a vinyl derivative. Then, the vinyl derivative is subjected to hydroboration with 9-borabicyclo[3.3.1]nonane (9-BBN), thereby obtaining a diol, whose structural formula is as follows: 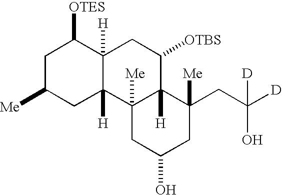
More specifically, the Wittig reaction may be carried out with Ph₃PCD₃Br and potassium hexamethyldisilazide (KHMDS) in THF, e.g., at 0° C. for two hours. The hydroboration may be carried out by treating the vinyl derivative with 9-BBN in THF, e.g., at 80° C. for one hour and then with hydrogen peroxide (H₂O₂) at room temperature for 12 hours. In this way, the diol can be obtained in a high yield.

Next, chemoselective oxidation of a secondary alcohol in the diol is performed by the Trost protocol (B. M. Trost, Y. Masuyama, Tetrahedron. Lett. 25. 173 (1984)) using ammonium molybdate (NH₄)₆Mo₇O₂₄.24H₂O) and hydrogen peroxide (H₂O₂) thereby giving rise to a keto alcohol whose structural formula is as follows: 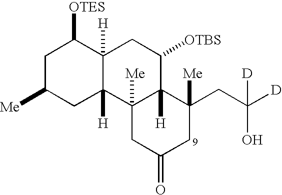
More specifically, this oxidation may be carried out with ammonium molybdate, tetrabutylammonium chloride (TBAC), K₂CO₃, and H₂O₂ in THF, e.g., at room temperature and then at 50° C. for seven hours. In the example in which this oxidation is actually performed by way of trial, a yield of the keto alcohol was 90% (for three steps).

Results of physical analysis of the keto alcohol were as follows:

[α]²⁹_{D}=−16.8° (c 1.00, CHCl₃); IR (CHCl₃) 3420, 2953, 1701, 1464,1381, 1256, 1057, 833, 756 cm⁻¹; ¹H NMR (CDCl₃, 270 MHz) δ 0.08 (s, 3 H), 0.09 (s, 3H), 0.60 (bq, J=8.2 Hz, 6 H), 0.89 (s, 9 H), 0.94-1.02 (m, 10 H, involving a broad triplet at 0.98, J=8.2 Hz), 1.11-1.80 (m, 9 H, involving singlets at 1.15, 1.18, and a doublet at 1.12, J=7.4 Hz), 1.80-2.20 (m, 3 H), 2.34 (dd, J=2.3, 12.4 Hz, 1 H), 2.40 (d, J=14.2 Hz, 1 H), 2.50 (dd, J=2.4, 12.4 Hz, 1 H), 3.75-3.80 (m, 1 H), 4.54-4.58 (m, 1 H); ¹³C NMR (67.8 MHz, CDCl₃) δ −4.40, −3.55, 5.05 (3 C), 7.16 (3 C), 18.02, 18.37, 21.14, 26.01 (3 C), 27.25, 28.36, 30.92, 36.39, 36.84, 38.43, 39.89, 42.35, 43.63, 43.68, 54.17, 56.49, 59.01, 68.14, 71.64, 212.27 (one peak missing); HRMS Calcd for C₂₇H₄₉D₂O₄Si₂ ([M-tBu]⁺); 497.3449. Found: 497.3444.

Then, a stereoselective construction of a quaternary asymmetric carbon center at the C-9 position was carried out, using the keto alcohol as a synthetic intermediate. A key conversion of the stereoselective construction is realized as follows:

The keto alcohol is treated with dimethyl carbonate [(MeO₂)C═O] and lithium tert-butoxide (tBuOLi) in THF and hexamethylphosphoramide (HMPA) at 75° C., whereby formation of carbonate and subsequent intramolecular acylation reaction smoothly occur to form a lithium enolate of β-keto lactone. Then, the lithium enolate is reacted with iodomethane (MeI) to give a methyl enol ether as a single product. A structural formula of the methyl enol ether is as follows: 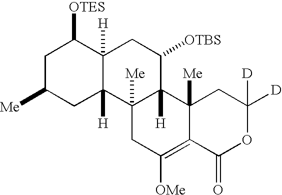
More specifically, the treatment may be carried out with (MeO)₂C═O, tBuOLi, and HMPA in THF, e.g., at 75° C. for four hours, and then with MeI, e.g., at room temperature for two hours. In the example in which this treatment was actually performed by way of trial, a yield of the methyl enol ether was 92%.

Results of physical analysis of the methyl enol ether were as follows:

[α]²⁹_{D}=+24.5° (c 0.84, CHCl₃); IR (CHCl₃) 2953, 2878, 1746, 1462,1254, 1221, 1128, 1096, 1042, 949, 833 cm⁻¹; ¹H NMR (CDCl₃, 270 MHz)δ 0.07 (s, 3 H), 0.08 (s, 3 H), 0.59 (bq, J=8.2 Hz, 6 H), 0.88 (s, 9 H), 0.94-1.00 (m, 10 H, involving a triplet at 0.97, J=8.2 Hz), 1.07 (s, 3 H), 1.15-1.72 (m, 15 H, involving a singlet at 1.22 and a doublet at 1.16, J=7.4 Hz), 1.91 (d, J=16.7 Hz, 1 H), 2.00-2.11 (m, 1 H), 2.38 (d, J=16.7 Hz, 1 H), 2.91 (d, J=14.2 Hz, 1 H), 3.69 (s, 3 H), 3.78 (bq, J=2.6 Hz, 1 H), 4.45-4.50 (m, 1 H); ¹³C NMR (67.8 MHz, CDCl₃)δ−4.49, −3.05, 5.08 (3 C), 7.19 (3 C), 16.93, 18.25, 21.21, 26.21 (3 C), 27.44, 31.82, 32.51, 32.55, 36.13, 36.78, 37.12, 38.54, 39.70, 41.46, 41.51, 54.09, 56.03, 68.07, 71.71, 110.86, 158.32, 168.20 (one peak missing); HRMS Calcd for C₃₃H₅₈D₂O₅Si₂ ([M]⁺); 594.4103. Found: 594.4105.

The methyl enol ether is further treated with tBuOLi in THF and 1,3-dimethyl-3,4,5,6-tetrahydro-2(1H)-pyrimidinone (DMPU). This treatment is followed by an addition of MeI. As a result, a compound which is targeted (hereinafter this compound is referred to as a target compound) is obtained as a single stereoisomer. The target compound has the following structural formula: 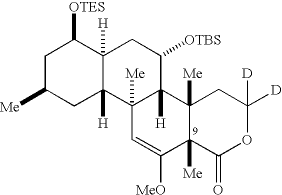
In the example in which this treatment and the addition were actually performed by way of trail, a yield of the target compound was 83%. The target compound has a methyl group newly introduced from β-side at the C-9 position highly stereoselectively. More specifically, this treatment to obtain the target compound is carried out with tBuOLi and DMPU at a temperature between 0° C. to room temperature for 1 hour and then with Mel at room temperature for two hours. In the example in which this treatment is actually carried out by way of trial, a yield of the target compound was 83%.

Results of physical analysis of the target compound were as follows:

[α]²⁹_{D}=+0.6° (c 0.85, CHCl₃); IR (CHCl₃) 3018, 2953, 1746, 1666, 1462, 1217, 1142, 1094, 1005, 976, 943, 835 cm⁻¹; ¹H NMR (CDCl₃, 270 MHz) δ 0.09 (s,3 H), 0.10 (s, 3 H), 0.59 (bq, J=7.9 Hz, 6 H), 0.89-1.03 (m, 22 H, involving singlets at 0.89, 1.03 and a triplet at 0.96, J=7.9 Hz), 1.14 (d, J=7.4 Hz, 3H), 1.17 (s, 3 H), 1.25-1.72 (m, 12 H, involving a singlet at 1.33), 2.00-2.13 (m, 1 H), 3.07 (bd, J=15.5 Hz, 1 H), 3.51 (s, 3 H), 3.75-3.80 (m, 1 H), 4.46-4.51 (m, 1 H), 4.79 (s, 1 H); ¹³C NMR (67.8 MHz, CDCl₃)δ −4.11, −3.83, 5.12 (3 C), 7.17. (3C), 18.02, 19.11, 19.87, 21.55, 25.99 (3 C), 27.56, 28.72, 31.22, 34.16, 37.21, 37.52, 38.68, 38.81, 39.54, 41.46, 49.22, 52.92, 54.81, 68.44, 71.65, 104.00, 152.37, 174.61 (one peak missing); HRMS Calcd for C₃₄H₆₀D₂O₅Si₂ ([M]⁺); 608.4259. Found: 608.4253.

Next, methyllithium (MeLi) is added to the target compound, thereby obtaining a primary alcohol. More specifically, this addition reaction is carried out with MeLi in ether (Et₂O), e.g., at 0° C. for one hour.

After the addition reaction, protection of the primary alcohol, is performed with a TBS group, thereby obtaining a compound whose structural formula is as follows (hereinafter this compound is referred to as a compound B): 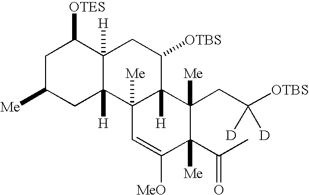
More specifically, this protection may be carried out with tert-butyldimethylsilyl chloride (TBSCl), triethylamine (Et₃N), and 4-dimethylaminopyridine (DMAP), in N,N-dimethylformamide (DMF), e.g., at room temperature for three hours. In the example in which this protection was carried out actually by way of trial, a yield of the compound B was 88% (for two steps).

Results of physical analysis of the compound B were as follows:

[α]²⁹_{D}=+18.5° (c 0.65, CHCl₃); IR (CHCl₃) 2953, 2856, 1699, 1668, 1472, 1387, 1254, 1219, 1142, 1057, 1007, 837 cm⁻¹; ¹H NMR (CDCl₃, 270 MHz)δ−0.03(s, 3 H), −0.02 (s, 3 H), 0.07 (s, 3 H), 0.09 (s, 3 H), 0.57 (bq, J=8.1 Hz, 6 H),0.81-0.98 (m, 31 H, involving singlets at 0.84, 0.89 and a triplet at 0.95, J=8.1 Hz), 1.10-1.72 (m, 17 H, involving singlets at 1.11, 1.13 and 1.31), 1.98-2.10 (m, 2 H), 2.22 (s, 3 H), 2.75 (d, J=14.2 Hz, 1 H), 3.49 (s, 3 H), 3.73-3.78 (m, 1 H), 4.41-4.46 (m, 1 H), 4.89 (s, 1 H); ¹³C NMR (67.8 MHz, CDCl₃)δ −5.24, −5.16, −4.31, −3.71, 5.12 (3 C), 7.19 (3 C), 18.05, 18.18, 19.32, 19.93, 20.20, 21.51, 25.96(3 C), 26.03, 26.08 (3 C), 27.70, 30.62, 31.48, 36.29, 37.27, 37.39, 38.80, 40.29, 41.88, 42.15, 51.80, 53.94, 59.90, 68.75, 71.93, 105.28, 154.78, 213.04; HRMS Calcd for C₄₁H₇₈D₂O₅Si₃ ([M]⁺); 738.5437. Found: 738.5441.

Next, from the resultant compound B, enol trifluoromethanesulfonate is formed. Then, the resultant enol trifluoromethanesulfonate is subjected to elimination reaction with diazabicyclo[5.4.0]undec-7-ene (DBU), thereby obtaining an alkyne segment, whose structural formula is as follows: 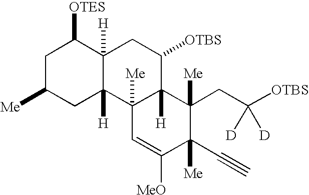
More specifically, these reactions may be carried out by treating the compound B with trifluoromethanesulfonic anhydride (Tf₂O) and 2,6-di-tert-butylpyridine (2,6-di-tBuPy), in dicholoroethane ((CH₂Cl)₂), e.g., at room temperature for three hours, and then with diazabicyclo[5.4.0]undec-7-ene(DBU), e.g., at 80° C. for three hours. In the example in which these reactions were actually carried out by way of trial, a yield of a compound resulted from these reactions was 81% (for 2 steps).

Results of physical analysis of the compound resulted from these reactions were as follows:

[α]³⁰_{D}=+6.7° (c 0.45, CHCl₃); IR (CHCl₃) 3308, 2856, 2361, 1668, 1254, 1217, 1057, 1007, 837 cm⁻¹; ¹H NMR (CDCl₃, 270 MHz)δ 0.00 (s, 3 H), 0.01 (s, 3 H), 0.09 (s, 3 H), 0.14 (s, 3 H), 0.58 (bq, J=8.2 Hz, 6 H), 0.87 (s, 9 H), 0.92 (s, 9 H), 0.96 (t, J=7.7 Hz, 9 H), 1.09 (s, 3 H), 1.13 (d, J=7.4 Hz, 3 H), 1.23-1.70 (m, 15 H, involving singlets at 1.26 and 1.32), 1.97 (d, J=13.8 Hz, 1 H), 1.97-2.10 (m, 1 H), 2.18 (s, 1 H), 2.58 (bd, J=13.8 Hz, 1 H), 3.58 (s, 3 H), 3.74-3.79 (m, 1 H), 4.41-4.45 (m, 1 H), 4.68 (s, 1 H); ¹³C NMR (67.8 MHz, CDCl₃) δ −5.17, −5.04, −4.42, −3.85, 5.00 (3C), 7.05 (3C), 17.94, 18.22, 20.09, 21.44, 25.66, 26. 00 (3C), 27.53, 31.32, 37.15, 37.29, 37.77, 38.65, 40.09, 41.59, 41.84, 46.40, 50.01, 55.04, 68.62, 70.69, 71.79, 88.74, 101.41, 153.22 (two peak missing); HRMS Calcd for C₄₁H₇₆D₂O₄Si₃ ([M]⁺); 720.5331. Found: 720.5319.

In the manner described above, the alkyne segment can be efficiently synthesized by using a deuterium kinetic isotope effect. Non-deuterated methyl ketone results in formation of a considerable amount of by-product (30% of which is not deuterated) along with the desired alkyne (66% of which is not deuterated). The by-product has the following structural formula:

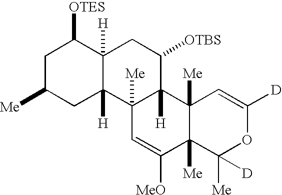

Results of physical analysis of the alkyne compound were as follows.

[α]³⁰^{D}=+25.0° (c 1.00, CHCl₃); IR (CHCl₃) 2953, 1665, 1472, 1379, 1254, 1140, 1055, 1067, 835, 760 cm⁻¹; ¹H NMR (CDCl₃, 270 MHz) δ 0.08 (s, 3 H), 0.11 (s, 3 H), 0.58 (bq, J=7.9 Hz, 6 H), 0.90-0.99 (m, 21 H, involving a singlet at 0.91 and a triplet at 0.96, J=7.9 Hz), 1.07 (s, 3 H), 1.13 (d, J=7.4 Hz, 3 H), 1.25 (s, 3 H), 1.29-1.72 (m, 12 H), 2.03 (br, 1 H), 3.47 (s, 3 H), 3.75 (s, 3 H), 4.46 (bs, 1 H), 4.76 (s, 1 H), 5.21-5.22 (m, 1 H), 6.23 (d, J=6.1 Hz, 1 H); ¹³C NMR(67.8 MHz, CDCl₃) δ −4.19, −3.65, 5.13 (3 C), 7.21 (3 C), 17.65, 17.88, 18.16, 19.08, 21.50, 25.28, 26.07 (3 C), 27.68, 31.22, 36.91, 37.05, 38.86, 39.64, 40.45, 41.36, 44.98, 45.06, 49.79, 54.14, 69.51, 71.93, 106.12, 111.89,112.09, 155.65; HRMS Calcd for C₃₅H₆₃DO₄Si₂ ([M]⁺); 605.4405. Found: 605.4449.

Mechanical analysis showed that the by-product is formed by 1,5-hydride shift from the compound B. The mechanism is illustrated below: 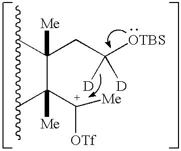
Based on this finding from the mechanical analysis, the inventors of the present invention accomplished an idea to use a deuterium kinetic isotope effect for this particular alkynylaton reaction in order to suppress the formation of the by-product. Indeed, the formation of the by-product was suppressed to less than 9% through this isotope effect. (cf. D. L. J Clive, M. Cantin, A. Khodabocus, X. Kong, Y. Tao, Tetrahedron, 49, 7917 (1993); D. L. J Clive, Y. Tao, A. Khodabocus, Y-J. Wu, A. G. Angoh, S. M. Benetee, C. N. Boddy, L. Bordeleau, D. Kellner, G. Kleiner, D. S. Middleton, C. J. Nicholas, S. R. Richardson, P. G. Venon, J. Am. Chem. Soc. 116, 11275 (1994); E. Vedejs, J. Little, J. Am. Chem. Soc. 124, 748 (2002))

Next, an amino alcohol fragment is synthesized starting from (R)-citronerol via the Jacobsen kinetic resolution protocol (H. Lebel, E. N. Jacobsen, Tetrahedron. Lett. 40, 7303 (1999)). A structural formula of the amino alcohol fragment is as follows: 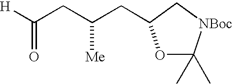
Results of physical analysis of the amino acid fragment were as follows:

(93:7 mixture) : [α]³⁰_{D}=−24.5° (c 1.50, CHCl₃); IR (neat) 2978, 2936, 2878, 1699, 1394, 1366, 1258, 1177 cm⁻¹; ¹H NMR (CDCl₃, 270 MHz) δ 1.04 (d, J=6.4 Hz, 3 H), 1.40-1.70 (m, 17 H, involving singlets at 1.47, 1.52 and 1.55), 2.13-2.34 (m, 2 H), 2.47-2.54 (m, 1 H), 2.95-3.10 (m, 1 H), 3.58-3.77 (m, 1 H), 4.04-4.15 (m, 1 H), 9.76 (s, 1 H); ¹³C NMR (67.8 MHz, CDCl₃) δ 20.51, 24.34, 25.25, 25.46, 26.37, 27.36, 28.51, 39.94, 50.76, 51.18, 71.43, 71.64, 79.40, 80.02, 92.92, 93.34, 151.67, 152.04, 202.04, involving peaks due to tautomer; HRMS Calcd for C₁₅H₂₈NO₄([M+H]⁺); 286.2018. Found: 286.2034.

Then, the alkyne segment and the thus synthesized amino alcohol fragment are coupled, thereby forming the alkynyl ketone. Then, a double bond is installed into the A-ring. A resultant compound after the installation of the double bond is subjected to bis-amianoacetalization to form a DEFG ring framework.

Firstly the coupling reaction of the alkyne segment and the amino alcohol segment is carried out with butyllithium (BuLi) in THF, followed by oxidation of adducts with 1,1,1-triacetoxy-1,1-dihydro-1,2-benziodoxol-3(1H)-one (DMP), thereby obtaining an alkynyl ketone, whose structural formula is as follows: 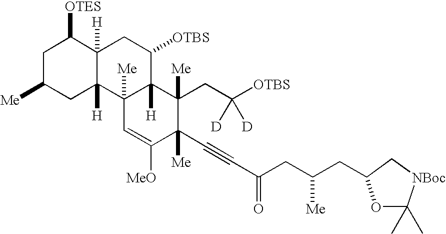
In the example in which these reactions were actually carried out, a yield of the alkynyl ketone was 82%.

More specifically, the coupling reaction may be carried out with BuLi in THF, e.g., at −30° C. for 30 minutes and then at −78° C. for one hour. The oxidation may be carried out with DMP and pyridine in CH₂Cl₂, e.g., at room temperature for two hours. In the example in which the coupling reaction was actually carried out by way of trial, a yield of the alkynyl ketone was 82%.

Results of physical analysis of the alkynyl ketone were as follows:

[α]³⁰^{D}=−2.0° (c 1.00, CHCl₃); IR (CHCl₃) 2955, 2930, 2880, 2858, 2210, 1703, 1672, 1464, 1391, 1366, 1256, 1219, 1177, 1059, 1009, 837 cm⁻¹; ¹H NMR (CDCl₃, 270 MHz) δ −0.02 (s, 3 H), −0.02 (s, 3 H), 0.08 (s, 3 H), 0.12 (s, 3 H), 0.57 (bq, J=7.9 Hz, 6 H), 0.84 (s, 9 H), 0.90 (s, 9 H), 0.95 (t, J=7.9 Hz, 9 H), 1.03 (d, J=6.6 Hz, 3 H), 1.08 (bs, 3 H), 1.12 (d, J=7.4 Hz,3 H), 1.24 (s, 3 H), 1.34 (s, 3 H), 1.38-1.69 (m, 27 H, involving a broad singlet at 1.47), 1.93 (d, J=14.2 Hz, 1 H), 1.96-2.01 (m, 1 H), 2.24-2.63 (m,4 H), 2.95-3.77 (m, 1 H), 3.54( s, 3 H), 3.60-3.78 (m, 2 H, involving a singlet at 3.78), 4.05-4.18 (m, 1 H), 4.41(bs, 1 H), 4.67 (s, 1 H); ¹³C NMR (67.8 MHz, CDCl₃)δ −5.21, −5.06, −4.26, −3.93, 5.00, 7.06, 17.93, 18.12, 20.10, 20.49, 21.46, 24.28, 25.15, 26.01, 26.24, 27.12, 27.32, 27.51, 28.46, 31.34, 37.19, 37.29, 38.01, 38.62, 39.80, 40.01, 41.88, 41.90, 47.17, 49.96, 51.11, 52.74, 52.96, 54.76, 68.45, 71.75, 71.99, 79.27, 79.81, 83.72, 92.68, 93.19, 98.53, 103.43, 151.61, 151.97, 152.39, 186.83, involving peaks due to tautomer; HRMS Calcd for C₅₆H₁₀₁D₂O₈Si₃([M]⁺); 1003.7115. Found: 1003.7146.

Next, hydrogenation of a triple bond of the resultant alkyl ketone is carried out. More specifically, the hydrogenation may be carried out with H₂ in the presence of platinum(V) oxide (PtO₂) in methanol (MeOH), e.g., at room temperature for 8 hours. Then, a resultant compound resulted from the hydrogenation is treated with aqueous acetic acid (AcOH), e.g., at 50° C. for five hours, thereby obtaining aminoacetal, whose structural formula is as follows:

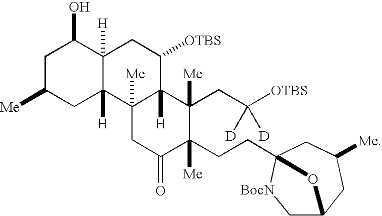

After that, the resultant aminoacetal is subjected to removal of two tert-butyldimethlsilyl (TBS) groups, then to selective oxidation of secondary hydroxyl groups, thereby obtaining a compound whose structural formula is as follows (hereinafter, this compound is referred to as a compound C:

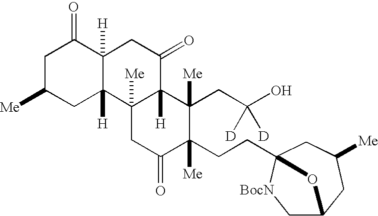

The removal of TBS groups is carried out with tetrabutylammonium fluoride (TBAF). More specifically, this removal may be carried out with TBAF in THF, e.g., at 70° C. for two hours. The selective oxidation is carried out with ammonium molybdate. More specifically, the selective oxidation may be carried out with ammonium molybdate, K₂CO₃ and H₂O₂ in THF, e.g., at 50° C. for three hours.

Next, a compound thus obtained by the selective oxidation is subjected to oxidation a primary alcohol with tetrapropylammonium perruthenate (TPAP), thereby obtaining an aldehyde. More specifically, the conversion to the aldehyde may be carried out with TPAP and NMO in CH₂Cl₂ by using a molecular sieve 4A, e.g., at room temperature for one hour.

Then, the thus obtained aldehyde is subjected to oxidation with sodium chloride (NaClO₂) thereby obtaining a carboxylic acid, whose structural formula is as follows:

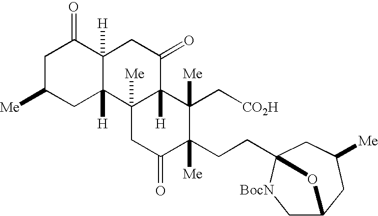

More specifically, this oxidation may be carried out with NaClO₂ and sodium dihydrogenphosphate (NaH₂PO₄) and 2-methyl-2-butene (Me₂C═CH(Me)) in aqueous, tert-butanol (tBuOH), e.g., at room temperature for one hour. In the example in which this oxidation was carried out actually by way of trial, a yield of the carboxylic acid was 61% (for six steps).

Results of physical analysis of the resultant compound (carboxylic acid) were as follows:

[α]³⁰_{D}=+5.9° (c 0.50, CHCl₃); IR (CHCl₃) 2956, 1703, 1396, 1290, 1244, 1176, 1120, 751 cm⁻¹; ¹H NMR (CDCl₃, 270 MHz)δ 0.90 (s, 3 H), 0.92 (s, 3 H), 1.01 (d, J=7.1 Hz, 3 H), 1.08-1.33 (m, 9 H, involving a singlet at 1.17, and a doublet at 1.28, J=8.2 Hz), 1.45 (s, 9 H), 1.53-1.61 (m, 2 H), 1.69-1.85 (m, 3H), 1.89-2.26 (m, 7 H, involving a doublet at 2.18, J=14.7), 2.44-2.68 (m, 6 H), 2.30 (s, 1 H), 3.14 (d, J=14.7 Hz, 1 H), 3.34-3.55 (m, 2 H), 3.62 (bd, J=2.1, 3 H), 4.41 (bs, 1 H); ¹³C NMR (67.8 MHz, CDCl₃) δ 16.20, 18.98, 19.06, 19.20, 19.30, 19.56, 21.53, 21.58, 22.55, 23.06, 24.12, 28.42, 28.48, 29.73, 30.05, 30.18, 31.50, 36.46, 36.65, 37.56, 41.13, 41.57, 42.87, 46.39, 47.06, 47.14, 47.17, 49.28, 49.36, 50.28, 50.90, 50.99, 51.06, 51.17, 51.23, 51.27, 54.71, 54.75, 62.40, 62.43, 72.30, 72.41, 79.03, 79.61, 94.17, 94.26, 151.61, 152.36, 171.89, 172.01, 208.22, 208.50, 208.67, 208.73, 211.61, 211.77, involving peaks due to tautomer; HRMS Calcd for C₃₅H₅₃NO₈ ([M]⁺); 615.3771. Found: 615.3777.

Regioselective introduction of a double bond into the A-ring can be successfully performed by using the Ito-Saegusa method. Firstly, the carboxylic acid is esterified with (trimethylsilyl)diazomethane (TMSCHN₂) in CH₂Cl₂. More specifically, this esterification may be carried out with TMSCHN₂ and MeOH in CH₂Cl₂, e.g., at room temperature for one hour. After that, a compound thus obtained via the esterification is treated with TMSCl and lithium hexamethyldisilazide (LHMDS) in THF, thereby solely producing trimethylsilyl enol ether of the ketone in the A-ring. More specifically, this treatment may be carried out, e.g., at −65° C. for one hour. A compound thus obtained via the treatment is reacted with palladium acetate (Pd(OAc)₂) in acetonitrile (CH₃CN), e.g., at 50° C. for two hours. The reaction gives a desired enone, whose structural formula is as follows: 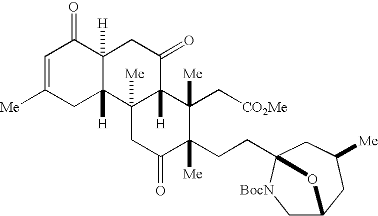
In the example in which this reaction was actually carried out by way of trial, a yield of the reaction was 96%.

Results of physical analysis of the enone were as follows:

[α]³⁰_{D}=+9.7° (c 0.45, CHCl₃); IR (CHCl₃) 3020, 1703, 1640, 1398, 1217, 1128, 756 cm⁻¹; ¹H NMR (CDCl₃, 270 MHz) δ 0.90 (d, J=1.1 Hz, 3 H), 0.92 (s, 3H), 1.09-1.31 (m, 9 H, involving a singlet at 1.17, and a doublet at 1.30, J=7.9 Hz), 1.45 (s, 9 H), 1.52-1.83 (m, 3 H), 2.00-2.24 (m, 10 H, involving a singletat 2.00, and a doublet at 2.20, J=14.8), 2.30-2.58 (m, 3 H), 2.76 (d, J=12.2 Hz, 1 H), 2.80-2.88 (m, 1 H), 3.01 (s, 1 H), 3.16 (d, J=14.8 Hz, 1 H), 3.34-3.55 (m, 2 H), 3.63 (bd, J=2.1 Hz, 3 H), 4.42 (bs, 1 H), 5.92 (s, 1 H); ¹³C NMR (67.8 MHz, CDCl₃) δ 16.73, 18.94, 19.35, 19.62, 21.54, 21.58, 22.57, 23.00, 24.12, 24.35, 28.42, 28.49, 30.09, 30.89, 31.55, 36.49, 36.70, 37.56, 41.09, 41.62, 43.14, 45.28, 45.42, 46.34, 46.37, 46.41, 48.43, 48.50, 50.90, 50.99, 51.09, 51.31, 51.82, 51.88, 54.73, 54.77, 62.07, 72.30, 72.42, 79.05, 79.61, 94.16, 94.22, 125.26, 151.63, 152.33, 159.86, 159.98, 171.84, 171.97, 197.69, 197.74, 207.49, 207.78, 211.37, 211.56, involving peaks due to tautomer; HRMS Calcd for C₃₅H₅₁NO₈ ([M]⁺); 613.3615.Found:613.3593.

Final bis-aminoacetalization, i.e., the construction of the DEFG rings culminating in the total synthesis of norzoanthamine, is carried out as follows: initially the enone is treated with aqueous acetic acid (AcOH) so as to obtain an iminium salt, whose structural formula is as follows: 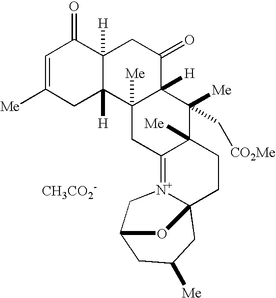
More specifically, this initial treatment may be carried out, e.g., at 100° C. for 24 hours.

Then, the resultant iminium salt is treated with aqueous trifluoroacetic acid (TFA), thereby producing an ammonium salt of norzoanthamine. A structural formula of the ammoniums salt of norzoanthamine is as follows: 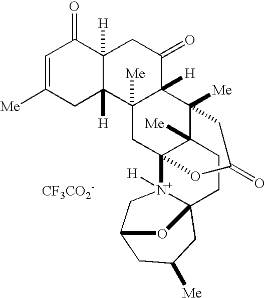
More specifically, this treatment may be carried out, e.g., at 100° C. for 24 hours.

Finally, the ammonium salt of norzoanthamine is subjected to desalination with basic alumina (Al₂O₃) in MeOH, thereby obtaining norzoanthamine, whose structural formula is as follow: 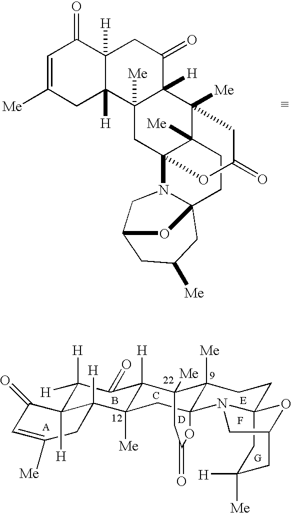
More specifically, the desalination may be carried out, e.g., at room temperature for one hour.

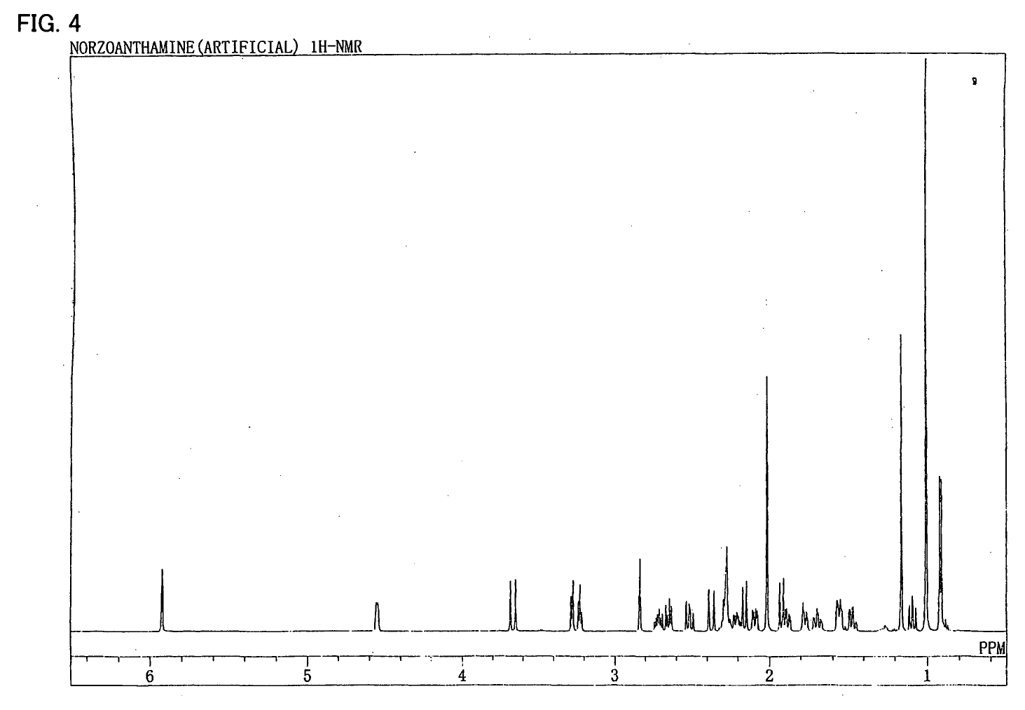

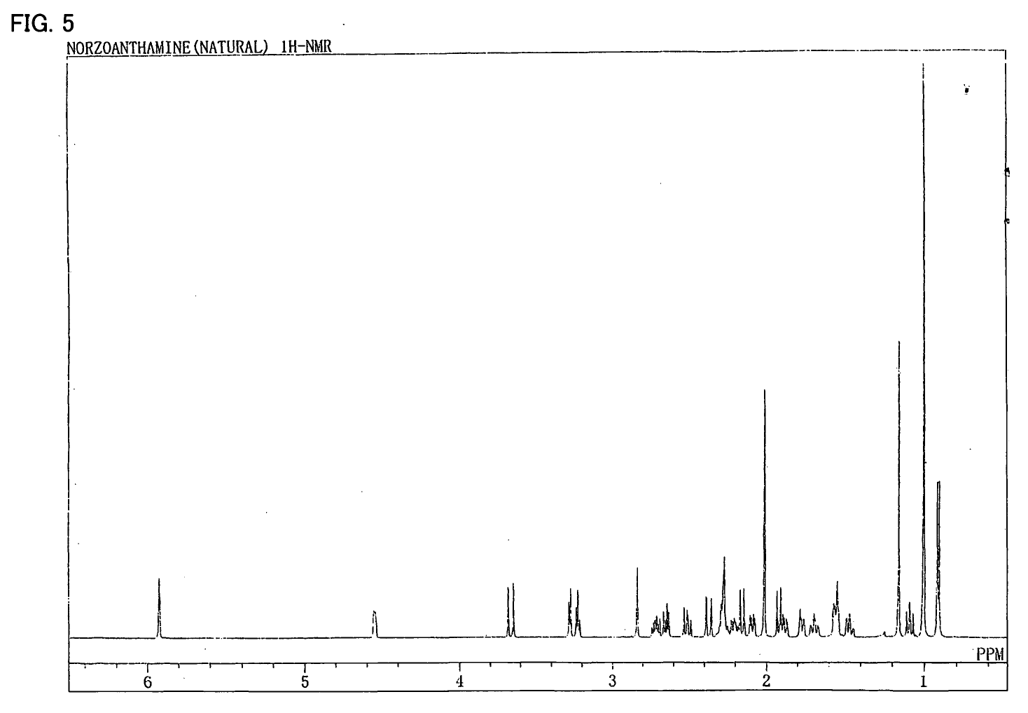

In the following, results of physical analysis of the thus synthesized norzoanthamine are shown, together with results of physical analysis of natural norzoanthamine. Moreover, FIGS. 4 and 5 illustrate NMR spectral analysis of the synthesized norzoanthamine and the natural norzoanthamine. mp 273-276° C.; artificial [α]²⁴_{D}=−6.0° (c 0.23, CHCl₃), natural[α] 24D=−6.2° (c 0.23, CHCl₃); CD artificial 313.4 nm (+1.57), 240.1 nm (−3.06), 227.2 nm (−2.07), 202.0 nm (−24.53) (0.0001 M, MeOH), natural 313.4 nm (1.32), 240.1 nm (−2.91), 226.9 nm (−1.85), 201.9 nm (−21.78) (0.0001 M, MeOH); UV artificial 233 nm (MeOH), natural 234 nm (MeOH); IR (CHCl₃) 3020, 2959, 1717, 1672, 1364,1248 cm⁻¹; ¹H NMR (CDCl₃, 270 MHz)δ 0.91 (d, J=6.7 Hz, 3 H), 1.01 (s, 6 H), 1.09 (dd, J=11.5, 12.7 Hz, 1 H), 1.16 (s, 3 H), 1.47 (dt, J=3.1, 12.5 Hz, 1 H), 1.54-1.58 (m, 2 H), 1.70 (bdt, J=3.7, 12.8 Hz, 1 H), 1.77 (bdt, J=3.4, 12.8 Hz, 1 H), 1.89 (bdt, J=4.9, 13.4 Hz, 1 H), 1.92 (d, J=14.0 Hz, 1 H), 2.02 (s, 3 H), 2.09 (dd, J=4.9, 12.7 Hz, 1 H), 2.16 (d, J=14.0 Hz, 1 H), 2.19-2.31 (m, 4 H), 2.37 (d, J=20.8 Hz, 1 H), 2.51 (dd, J=11.6, 14.6 Hz, 1 H), 2.65 (dd, J=6.1, 14.6 Hz, 1 H), 2.72 (bdt, J=6.1, 11.6 Hz, 1 H), 2.84 (s, 1 H), 3.23 (dd, J=6.1, 6.7 Hz, 1 H), 3.28 (d, J=6.7, 1 H), 3.67 (d, J=20.8 Hz, 1 H), 4.55-4.56 (m, 1 H), 5.92 (bs, 1 H); ¹³C NMR (67.8 MHz, CDCl₃) δ 18.40, 18.47, 21.09, 21.82, 22.95, 23.66, 24.30, 29.93, 31.98, 35.88, 36.47, 38.87, 39.80, 39.89, 41.88, 42.42, 44.38, 46.44, 47.15, 53.15, 59.12, 74.22, 89.98, 101.52, 125.62, 159.82, 172.43, 198.46, 208.98; HRMS Calcd for C₂₉H₃₉NO₅ ([M]⁺); 481.2828. Found: 481.2841.

In the example, the overall synthesis of norzoanthamine was carried out in 41 steps in total, an overall yield of the synthesized norzoanthamine was 3.5%, and an average yield of each step is 92%. The synthesized norzoanthamine is identical in all respects with naturally occurring norzoanthamine, including spectroscopic characteristics (¹H and ¹³C NMR spectra, infrared spectroscopy, and mass spectra), circular dichroism (CD), and optical rotation. In terms of optical rotation, the synthesized norzoanthamine has [α]²⁴_{D} −6.0 (c 0.23, CHCl₃), while the natural occurring norzoanthamine has [α]²⁴_{D} −6.2 (c 0.23, CHCl₃).

The absolute structure of norzoanthamine is verified by the present total synthesis. The chemistry described here not only offers a solution to a formidable synthetic challenge but also opens a completely chemical avenue to norzoanthamine, other naturally occurring zoanthamine alkaloids, and synthetic, designed norzoanthamine derivatives.

The invention being thus described, it will be obvious that the same way may be varied in many ways. Such variations are not to be regarded as a departure from the spirit and scope of the invention, and all such modifications as would be obvious to one skilled in the art are intended to be included within the scope of the following claims.

For instance, the compounds used in the respective reactions in the embodiment are discussed merely for exemplification, and may be replaced with other compounds having similar function as appropriate. Moreover, the present invention is not limited to the reaction time, reaction temperature, and the other conditions mentioned in the embodiment.

1. A method of producing a zoanthamine alkaloid, comprising the steps of:
converting a first compound to a second compound;
converting the second compound to a third compound;
converting the third compound to a fourth compound;
converting the fourth compound to a fifth compound;
converting the fifth compound to a sixth compound; and
converting the sixth compound to the zoanthamine alkaloid,
the first compound represented by: 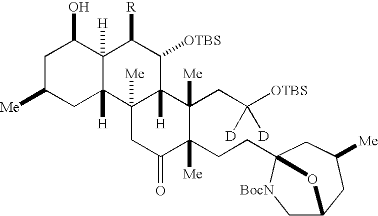
the second compound represented by: 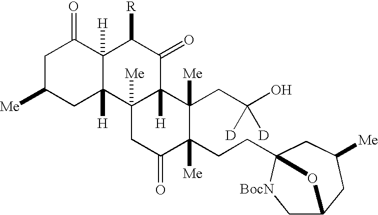
the third compound represented by: 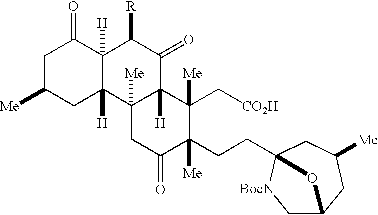
the fourth compound represented by: 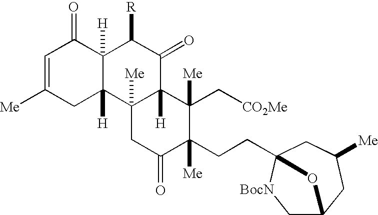
the fifth compound represented by: 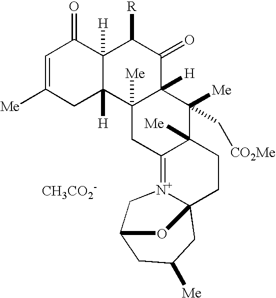
the sixth compound represented by: 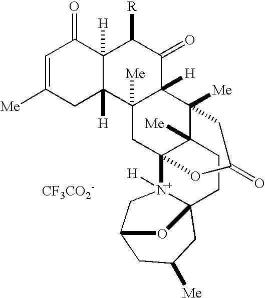
where R is H or CH₃, D is a deuterium, TBS is a tert-butyldimethylsilyl group, Boc is a tert-butoxycarbonyl group, and Me is a methyl group.

2. A method of producing a zoanthamine alkaloid, comprising the steps of:
removing two tert-butyldimethylsilyl groups from a first compound and selectively oxidizing secondary hydroxyl groups of the first compound, so as to obtain a second compound;
oxidizing the second compound to an aldehyde and oxidizing the aldehyde to a carboxylic acid, so as to obtain a third compound;
esterifying the third compound and introducing a double bond into an A-ring of the esterified third compound, so as to obtain a fourth compound;
producing an iminium salt of the fourth compound, so as to obtain a fifth compound;
producing an ammonium salt of the fifth compound, so as to obtain a sixth compound; and
desalinating the sixth compound, so as to obtain the zoanthamine alkaloid,
the first compound represented by: 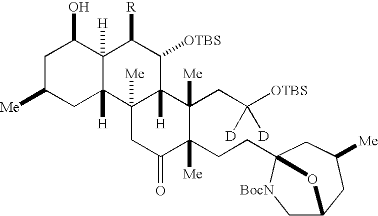
the second compound represented by: 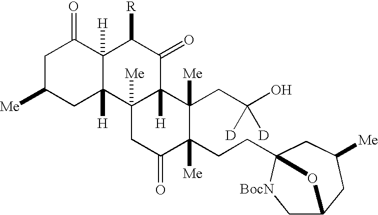
the third compound represented by: 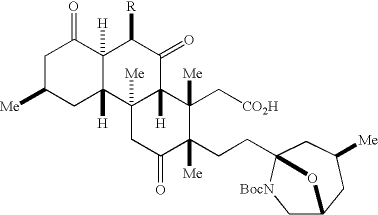
the fourth compound represented by: 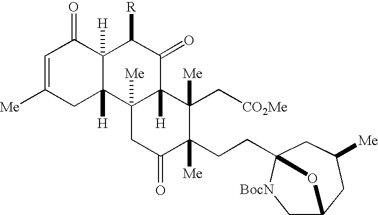
the fifth compound represented by: 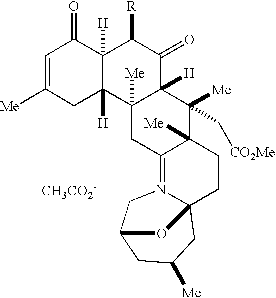
the sixth compound represented by: 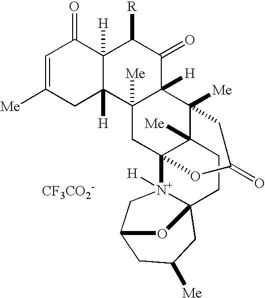
where R is H or CH₃, D is a deuterium, TBS is a tert-butyldimethylsilyl group, Boc is a tert-butoxycarbonyl group, and Me is a methyl group.

3. A method as set forth in claim 1, wherein:
the zoanthamine alkaloid is norzoanthamine.

4. A method as set forth in claim 1, wherein:
the zoanthamine alkaloid is zoanthamine.

5. A method of producing a zoanthamine alkaloid, comprising the steps of:
converting a seventh compound to an eighth compound;
converting the eighth compound to a ninth compound;
converting the ninth compound to a tenth compound;
converting the tenth compound to an eleventh compound;
converting the eleventh compound to a twelfth compound; and
converting the twelfth compound to a thirteenth compound,
the seventh compound represented by: 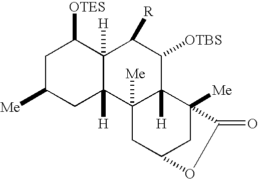
the eighth compound represented by: 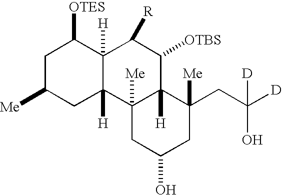
the ninth compound represented by: 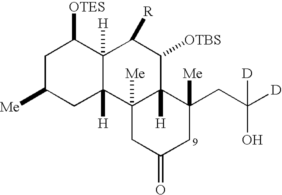
the tenth compound represented by: 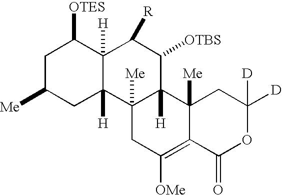
the eleventh compound represented by: 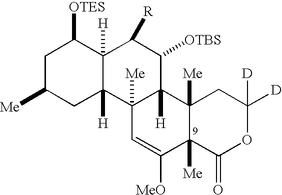
the twelfth compound represented by: 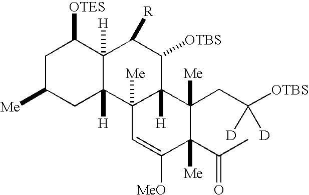
the thirteenth compound represented by: 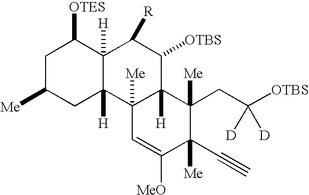
where R is H or CH₃, D is a deuterium, TBS is a tert-butyldimethylsilyl group, TES is a triethylsilyl group, and Me is a methyl group.

6. A method of producing a zoanthamine alkaloid, comprising the steps of:
subjecting a seventh compound to (a) reduction, (b) a Wittig reaction with a compound containing a deuterium, and (c) hydroboration, so as to obtain an eighth compound;
subjecting the eighth compound to oxidation so as to obtain a ninth compound;
forming a carbonate of the ninth compound, and subjecting the carbonate of the ninth compound to an intramolecular acylation reaction and subsequent methylation reaction, so as to obtain a tenth compound;
introducing a methyl group at a C-9 position of the tenth compound so as to obtain an eleventh compound;
adding a methyl group to a carbon that is bound with an oxygen with which deuteriums are bound, so as to obtain a twelfth compound; and
converting a methyl ketone of the twelfth compound to a triple bond, so as to obtains a thirteen compound,
the seventh compound represented by: 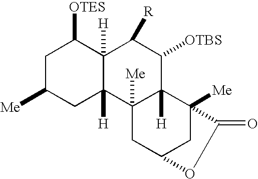
the eighth compound represented by: 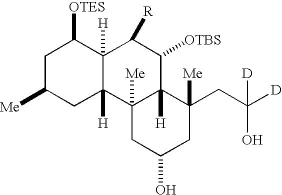
the ninth compound represented by: 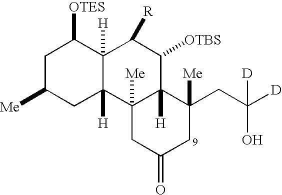
the tenth compound represented by: 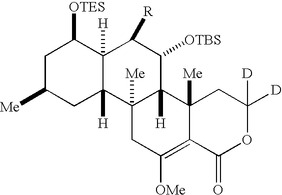
the eleventh compound represented by: 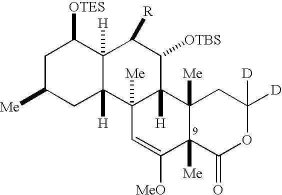
the twelfth compound represented by: 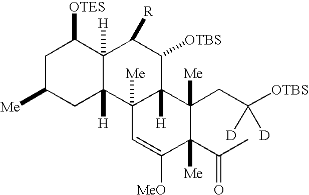
the thirteenth compound represented by: 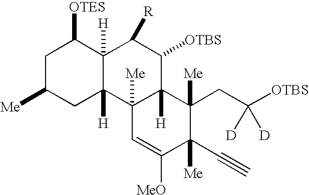
where R is H or CH₃, D is a deuterium, TBS is a tert-butyldimethylsilyl group, TES is a triethylsilyl group, and Me is a methyl group.

7. A method as set forth in claim 5, wherein:
the zoanthamine alkaloid is norzoanthamine.

8. A method as set forth in claim 5, wherein:
the zoanthamine alkaloid is zoanthamine.

9. An intermediate represented by: 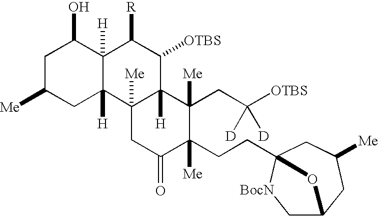
where R is H or CH₃, TBS is a tert-butyldimethylsilyl group, Boc is a tert-butoxycarbonyl group, and Me is a methyl group.

10. An intermediate represented by: 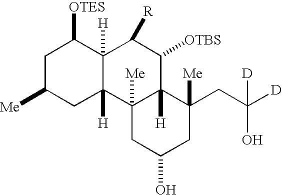
where R is H or CH₃, D is a deuterium, TBS is a tert-butyldimethylsilyl group, TES is a triethylsilyl group, and Me is a methyl group.

11. An intermediate represented by: 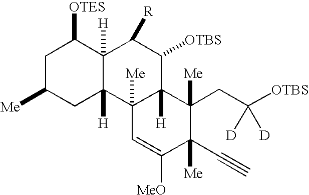
where R is H or CH₃, D is a deuterium, TBS is a tert-butyldimethylsilyl group, TES is a triethylsilyl group, and Me is a methyl group.

12. A method as set forth in claim 2, wherein:
the zoanthamine alkaloid is norzoanthamine.

13. A method as set forth in claim 2, wherein:
the zoanthamine alkaloid is zoanthamine.

14. A method as set forth in claim 6, wherein:
the zoanthamine alkaloid is norzoanthamine.

15. A method as set forth in claim 6, wherein:
the zoanthamine alkaloid is zoanthamine.
